(19) United States
(12) Patent Application Publication
(10) Pub. No.: US 2020/0041492 A1
(43) Pub. Date: Feb. 6, 2020

(54) BIOMARKERS FOR CELLULAR SENESCENCE

(71) Applicants: RIJKSUNIVERSITEIT GRONINGEN, Groningen (NL); ACADEMISCH ZIEKENHUIS GRONINGEN, Groningen (NL)

(72) Inventor: MARCO DEMARIA, GRONINGEN (NL)

(21) Appl. No.: 16/492,410

(22) PCT Filed: Mar. 9, 2018
(86) PCT No.: PCT/NL2018/050148

§ 371 Date: Sep. 9, 2019

(30) Foreign Application Priority Data
Mar. 9, 2017 (EP) 17160165.1

(51) Int. Cl. G01N 33/50; A61K 47/68

(52) U.S. Cl. CPC G01N 33/5091; A61K 47/6829; A61K 47/6849

(57) ABSTRACT

The invention relates to biomarkers and uses thereof, in particular to a set of proteins or mRNAs that provides a significant indication as to whether a cell is senescent or not. Provided is the use of a biomarker panel comprising six or more polypeptides, or their encoding mRNA's, wherein the panel comprises at least the biomarkers TSPAN13, GDNF, C2CD5, SUSD6, BCL2L2, PLK3, or a variant or fragment thereof, as a biomarker set for cellular senescence. Also provided is a senescent cell detection kit for detecting senescent cells, and a drug conjugate for killing a senescent cell.

The invention relates to biomarkers and uses thereof. In particular, the invention relates to set of proteins or mRNAs (biomarker array) that provides a significant indication as to whether a cell is senescent or not. It also relates to a drug conjugate for killing a senescent cell, and to pharmaceutical compositions comprising the same.

Cellular senescence is a change in the cellular state, whereby the cell cannot replicate anymore. Although cellular senescence is linked to ageing, as more cells fail to replicate with telomere shortening, it is by no means solely regulated by it. It should thus not be confused with ageing. Cells can also be induced into a senescent state via the activation of oncogenes, cell-cell fusions, through DNA damage and/or in response to elevated Reactive Oxygen Species (ROS), independent of ageing.

Although senescence cells cannot replicate, they are still metabolically active and commonly adopt an immunogenic phenotype that contributes to many age related diseases, including type 2 diabetes and atherosclerosis. The damaging effects of these senescence associated secretory phenotypes (SASP) has in turn stimulated a number of activities surrounding cellular senescence, including the identification of novel compounds to eliminate senescence cells and improve health in the elderly as well as the discovery of novel biomarkers to identify cellular senescence.

When a cell enters into a senescent state it is characterized by a number of non-unique features, including changes in morphology, activation of enzymes, chromatin remodeling and transcriptional alterations. Senescent cells effect embryogenesis, tumor suppression, wound healing and have a pathological control on age related diseases. A universal biomarker of cellular senescence will provide a robust tool to identify the likely onset of a number of age-related diseases, and may provide a pathway for early intervention strategies to prevent their development. Current studies have shown that pharmacologically removing senescent cells in ageing mice significantly extends health- and life-span. A number of recently formed companies are currently developing drugs that can be used to treat humans. A robust biomarker would assist physicians in determining whether a patient is at risk in developing a number of age-related diseases linked to increase cellular senescence. Furthermore, it would be desirable to identify new targets that can serve as a basis for a pharmacological intervention e.g. to postpone ageing and age-related diseases.

However, any of the currently available methods/assays to measure such biomarkers have significant drawbacks e.g. they are non-specific, can identify a number of other cellular perturbations, and/or are strongly cell type and stress-dependent.

The present inventors therefore set out to provide a novel biomarker panel that can identify any type of senescent cell without the need for further characterizations and validations. Surprisingly, through extensive comparisons of many different whole-transcriptome datasets, they identified a list of 37 transcriptome markers (see Table 1) that provides a universal array which is highly specific for the senescent cell state.

In one aspect, the invention provides the use of a set of at least five polypeptides, or their encoding mRNA's, selected from the group consisting of TSPAN13, GDNF, C2CD5, CNTLN, FAM214B, PATZ 1, PLXNA3, STAG1, SUSD6, TOLLIP, TRDMT1, ZBTB7A, ARID2, B4GALT7, BCL2L2, CHMP5, CREBBP, DDA1, DYNLT3, EFNB3, ICE1, MEIS1, NOL3, PCIF1, PDLIM4, PDS5B, PLK3, RAI14, RHNO1, SCOC, SLC16A3, SMO, SPIN4, TAF13, TMEM87B, MTCYB, UFM1 and ZNHIT1, or a variant or fragment thereof, as a biomarker panel (also herein referred to as “core signature”) for cellular senescence.

TABLE 1
transcriptome markers that highly specific for the senescent cell state.

| Gene_Name | Ensembl_ID | Gene_ID_NCBI | Synonyms |
|---|---|---|---|
| ARID2 | ENSG00000189079 | 196528 | p200, BAF200, KIAA1557, |
| | | | DKFZp686G052, FLJ30619 |
| B4GALT7 | ENSG00000027847 | 11285 | EDSP1, XGPT1, XGALT-1, |
| | | | XGALT1, EDSSLA, |
| | | | beta4Gal-T7 |
| BCL2L2 | ENSG00000129473 | 599 | KIAA0271, BCL2-L-2, |
| | | | BCLW, PPP1R51, BCL-W |
| C2CD5 | ENSG00000111731 | 9847 | CDP138, KIAA0528 |
| CHMP5 | ENSG00000086065 | 51510 | PNAS-2, Vps60, C9orf83, |
| | | | HSPC177, SNF7DC2, CGI-34 |
| CNTLN | ENSG00000044459 | 54875 | C9orf39, FLJ20276, |
| | | | bA340N12.1, C9orf101 |
| CREBBP | ENSG00000005339 | 1387 | CBP, KAT3A, RTS, RSTS |
| DDA1 | ENSG00000130311 | 79016 | C19orf58, MGC2594, PCIA1 |
| DYNLT3 | ENSG00000165169 | 6990 | TCTE1XL, TCTEX1L, RP3, |
| | | | TCTE1L |
| EFNB3 | ENSG00000108947 | 1949 | EFL6, LERK-8, EPLG8, |
| | | | LERK8 |
| FAM214B | ENSG00000005238 | 80256 | FLJ11560, bA182N22.6, |
| | | | KIAA1539 |
| GDNF | ENSG00000168621 | 2668 | HFB1-GDNF, ATF1, |
| | | | HSCR3, ATF2, ATF |
| ICE1 | ENSG00000164151 | 23379 | KIAA0947 |
| MEIS1 | ENSG00000143995 | 4211 | |
| NOL3 | ENSG00000140939 | 8996 | MYP, ARC, GN NOP, NOP, |
| | | | FCM, NOP30, CARD2, ARC |
| PATZ1 | ENSG00000100105 | 23598 | ZBTB19, dJ400N23, |
| | | | ZNF278, RIAZ, MAZR, ZSG, |
| | | | PATZ |
| PCIF1 | ENSG00000100982 | 63935 | bA465L10.1, PPP1R121, |
| | | | C20orf67 |
| PDLIM4 | ENSG00000131435 | 8572 | RIL |
| PDS5B | ENSG00000083642 | 23047 | AS3, GN KIAA0979, AS3, |
| | | | KIAA0979, FLJ23236, |
| | | | APRIN, CG008 |
| PLK3 | ENSG00000173846 | 1263 | PLK-3, CNK, FNK, PRK |
| PLXNA3 | ENSG00000130827 | 55558 | PLXN4, Plxn3, |
| | | | HSSEXGENE, XAP-6, |
| | | | PLXN3, SEX, 6.3 |
| RAI14 | ENSG00000039560 | 26064 | DKFZp564G013, NORPEG, |
| | | | KIAA1334, RAI13 |
| RHNO1 | ENSG00000171792 | 83695 | HKMT1188, C12orf32, |
| | | | MGC13204, RHINO |
| SCOC | ENSG00000153130 | 60592 | UNC-69, HRIHFB2072, |
| | | | SCOCO |
| SLC16A3 | ENSG00000141526 | 9123 | MCT-3, MCT 3, MCT3, hsa- |
| | | | mir-6787, MCT-4, MCT4, |
| | | | MCT 4 |
| SMO | ENSG00000128602 | 6608 | SMOH, CRJS, FZD11, Gx |
| SPIN4 | ENSG00000186767 | 139886 | TDRD28, FLJ44984 |
| STAG1 | ENSG00000118007 | 10274 | SA-1, SA1, SCC3A |
| SUSD6 | ENSG00000100647 | 9766 | DRAGO, GN KIAA0247, |
| | | | KIAA0247, DRAGO |
| TAF13 | ENSG00000197780 | 6884 | TAFII18, TAFII-18, |
| | | | TAF(II)18, TAF2K |
| TMEM87B | ENSG00000153214 | 84910 | FLJ14681 |
| TOLLIP | ENSG00000078902 | 54472 | IL-1RAcPIP |
| TRDMT1 | ENSG00000107614 | 1787 | DMNT2, PUMET, DNMT2, |
| | | | MHSAIIP, RNMT1 |
| TSPAN13 | ENSG00000106537 | 27075 | NET-6, NET6, TM4SF13 |
| UFM1 | ENSG00000120686 | 51569 | BM-002, bA131P10.1, |
| | | | C13orf20 |
| ZBTB7A | ENSG00000178951 | 51341 | ZBTB7, FBI1, LRF, |
| | | | DKFZp547O146, FBI-1, |
| | | | pokemon, ZNF857A, TIP21 |
| ZNHIT1 | ENSG00000106400 | 10467 | CG1I, CGBP1, ZNFN4A1, |
| | | | H_DJ0747G18.14 |

More in particular, the invention provides the use of a panel comprising six polypeptides, or their encoding mRNA's, wherein the panel comprises the biomarkers TSPAN13, GDNF, C2CD5, SUSD6, BCL2L2, PLK3, or a variant or fragment thereof, as a biomarker set (also herein referred to as “minimal core signature”) for cellular senescence.

If so desired, this minimal core signature as provided herein may be supplemented with one or more additional cellular senescence markers. For example, the biomarker set comprises at least seven, more preferably at least eight or even more polypeptides or mRNA's comprising the six biomarkers of the minimal core signature, and one or more selected from the group consisting of CTLN, FAM214B, PATZ 1, PLXNA3, STAG1, TOLLIP, TRDMT1, ZBTB7A, ARID2, B4GALT7, CHMP5, CREBBP, DDA1, DYNLT3, EFNB3, ICE1, MEIS1, NOL3, PCIF1, PDLIM4, PDS5B, RAI14, RHNO1, SCOC, SLC16A3, SMO, SPIN4, TAF13, TMEM87B, UFM1 and ZNHIT1.

In one embodiment, the set comprises the biomarkers TSPAN13, GDNF, C2CD5, SUSD6, BCL2L2, PLK3, and one or both of DYNLT3 and PLXNA3.

Particularly preferred is the use of a set comprising at least five polypeptides, or their encoding mRNA's, selected from the group consisting of GDNF, C2CD5, CNTLN, FAM214B, PATZ 1, PLXNA3, STAG1, SUSD6, TOLLIP, TRDMT1 and ZBTB7A, or a variant or fragment thereof, as a biomarker panel for cellular senescence. Preferably, at least the marker GDNF is included. More preferably, at least the marker TSPAN13 is included. For example, the set comprises or consists of TSPAN 13, GDNF, PATZ 1, PLXNA3, STAG1, and SUSD6, or TOLLIP, TRDMT1, ZBTB7A, C2CD5 and CNTLN, or FAM214B, GDNF, PATZ 1, PLXNA3 and STAG1.

In one embodiment, the set comprises at least one or more biomarker(s) that is/are upregulated in a senescent cell. These include GDNF, FAM214B, PLXNA3, SUSD5, TOLLIP, ZBTB7A, B4GALT7, BCL2L2, CHMP5, DDA1, DYNLT3, NOL3, PDLIM4, PLK3, RAI14, SCOC, SLC16A3, TAF13, TMEM87B, TSPAN13, UFM1 and ZNHIT1. Preferred are TSPAN 13, GDNF, FAM214B, PLXNA3, SUSD6, TOLLIP and/or ZBTB7A, or a variant or fragment thereof. More preferred are TSPAN13, GDNF, BCL2L2 PLXNA3, SUSD6 and DYNLT3.

In a separate embodiment, the invention provides the use of TSPAN13 polypeptide or its encoding mRNA, or a variant or fragment thereof, as biomarker panel for cellular senescence, either as single biomarker or being part of a multi-biomarker panel.

According to the present invention, a set of molecular biomarkers highly specific for the senescent cell state can include any product expressed by these genes, including variants thereof, e.g. expressed mRNA or protein, splice variants, co- and post-translationally modified proteins, polymorphic variants etc.

In one embodiment, the biomarker panel comprises a set of polypeptides, polypeptide variants or polypeptide fragments.

The terms “polypeptide fragment” or “fragment”, when used in reference to a polypeptide, refers to a polypeptide in which amino acid residues are absent as compared to the full-length polypeptide itself, but where the remaining amino acid sequence is usually identical to the corresponding positions in the reference polypeptide. Such deletions can occur at the amino-terminus or carboxy-terminus of the reference polypeptide, or alternatively both. Fragments typically are at least 5, 6, 8 or 10 amino acids long, at least 14 amino acids long, at least 20, 30, 40 or 50 amino acids long, at least 75 amino acids long, or at least 100, 150, 200, 300, 500 or more amino acids long.

A fragment can retain one or more of the biological activities of the reference polypeptide. In some embodiments, a fragment can comprise a domain or feature, and optionally additional amino acids on one or both sides of the domain or feature, which additional amino acids can number from 5, 10, 15, 20, 30, 40, 50, or up to 100 or more residues. Further, fragments can include a sub-fragment of a specific region, which sub-fragment retains a function of the region from which it is derived.

In another embodiment, a biomarker panel comprises a set of mRNA's.

The invention also provides a method of detecting a senescent cell in a test sample, the method comprises (in vitro) detecting the expression in the sample, of at least the set of senescent cell biomarkers according to the invention, wherein an increased level of expression of TSPAN13, C2CD5, GDNF, PLXNA3, SUSD6, BCL2L2, or a variant or fragment thereof, relative to the level of expression detected in a reference sample is an indication of the presence of a senescent cell in the sample.

In a specific embodiment, the method comprises (in vitro) detecting the expression in the sample of at least TSPAN13, wherein an increased level of TSPAN13 expression, or a variant or fragment thereof, relative to the level of expression detected in a reference sample is an indication of the presence of a senescent cell in the sample.

Preferably, the expression level of one or more senescence biomarkers (polypeptide or mRNA) is normalized to a reference or “housekeeping” gene (product) known in the art, such as tubulin or actin.

As used herein, the term ‘senescent cell’ refers to a cell showing at least 2-fold increased beta-galactosidase activity, and reduced proliferation e.g. as evidenced by incorporation of 5-ethynyl-2′-deoxyuridine (EdU) into de novo DNA.

The test subject can be an experimental animal or a human. The test sample is for example a bodily sample taken from a test subject. Preferably, the sample comprises blood, plasma, serum, spinal fluid, urine, sweat, saliva, tears, breast aspirate, prostate fluid, seminal fluid, vaginal fluid, stool, cervical scraping, cytes, amniotic fluid, intraocular fluid, mucous, moisture in breath, animal tissue, cell lysates, tumour tissue, hair, skin, buccal scrapings, nails, bone marrow, cartilage, prions, bone powder, ear wax, or combinations thereof. The test sample can be an ex vivo sample or an in vitro sample.

Expression of a biomarkers described in this invention may be assessed by any of a wide variety of well known methods for detecting expression of a transcribed nucleic acid or protein. Non-limiting examples of such methods include immunological methods for detection of secreted, cell-surface, cytoplasmic, or nuclear proteins, protein purification methods, protein function or activity assays, nucleic acid hybridization methods, nucleic acid reverse transcription methods, and nucleic acid amplification methods.

In one embodiment, expression of a biomarker set is assessed using antibodies (e.g. a radio-labeled, chromophore-labeled, fluorophore-labeled, or enzyme-labeled antibody), antibody derivatives (e.g. an antibody conjugated with a substrate or with the protein or ligand of a protein-ligand pair {e.g. biotin-streptavidin}), or antibody fragments (e.g. a single-chain antibody, an isolated antibody hypervariable domain, etc.) which bind specifically with a biomarker protein or fragment thereof, including a biomarker protein which has undergone either all or a portion of post-translational modifications to which it is normally subjected in the tumor cell (e.g. glycosylation, phosphorylation, methylation etc.).

Hence, numerous methods and devices are well known to the skilled artisan for the detection and analysis of the biomarker polypeptides of the presently disclosed subject matter. With regard to polypeptides or proteins in subject test samples, mass spectrometry and/or immunoassay devices and methods can be used, although other methods are well known to those skilled in the art (for example, the measurement of marker RNA levels). See, e.g., U.S. Pat. Nos. 6,143,576; 6,113,855; 6,019,944; 5,985,579; 5,947,124; 5,939,272; 5,922,615; 5,885,527; 5,851,776; 5,824,799; 5,679,526; 5,525,524; and 5,480,792. These devices and methods can utilize labeled molecules in various sandwich, competitive, or non-competitive assay formats, to generate a signal that is related to the presence or amount of an analyte of interest. Additionally, certain methods and devices, such as biosensors and optical immunoassays, can be employed to determine the presence or amount of analytes without the need for a labeled molecule. See, e.g., U.S. Pat. Nos. 5,631,171; and 5,955,377.

In certain preferred embodiments of the presently disclosed subject matter, the biomarker peptides are analyzed using an immunoassay. The presence or amount of a peptide marker can be determined using antibodies or fragments thereof specific for each polypeptide of the senescence biomarker panel, and detecting specific binding. For example, in some embodiments, the antibody specifically binds a peptide of Table 1, which includes antibodies that bind the full-length peptide as well. In some embodiments, the antibody is a monoclonal antibody.

Any suitable immunoassay can be utilized, for example, enzyme-linked immunoassays (ELISA), radioimmunoassays (RIAs), competitive binding assays, and the like. Specific immunological binding of the antibody to the marker can be detected directly or indirectly. Direct labels include fluorescent or luminescent tags, metals, dyes, radionuclides, and the like, attached to the antibody. Indirect labels include various enzymes well known in the art, such as alkaline phosphatase, horseradish peroxidase and the like.

The use of immobilized antibodies or fragments thereof specific for the markers is also contemplated by the present subject matter. The antibodies can be immobilized onto a variety of solid substrates, such as magnetic or chromatographic matrix particles, the surface of an assay plate (such as microtiter wells), pieces of a solid substrate material (such as plastic, nylon, paper), and the like. An assay strip can be prepared by coating the antibody or a plurality of antibodies in an array on solid support. This strip can then be dipped into the test biological sample and then processed quickly through washes and detection steps to generate a measurable signal, such as for example a colored spot.

In some embodiments, mass spectrometry (MS) analysis can be used alone or in combination with other methods (e.g., immunoassays) to determine the presence and/or quantity of the one or more polypeptide biomarkers of interest in a biological sample. In some embodiments, the MS analysis comprises matrix-assisted laser desorption/ionization (MALDI) time-of-flight (TOF) MS analysis, such as for example direct-spot MALDI-TOF or liquid chromatography MALDI-TOF mass spectrometry analysis. In some embodiments, the MS analysis comprises electrospray ionization (ESI) MS, such as for example liquid chromatography (LC) ESI-MS. Mass analysis can be accomplished using commercially-available spectrometers, such as for example triple quadrupole mass spectrometers. Methods for utilizing MS analysis, including MALDI-TOF MS and ESI-MS, to detect the presence and quantity of biomarker peptides in biological samples are known in the art. See for example U.S. Pat. Nos. 6,925,389; 6,989,100; and 6,890,763 for further guidance. In some embodiments, the MS analysis can be utilized to identify specific polypeptide sequences and corresponding proteins and amounts compared to controls. However, MS analysis can also be utilized with the methods of the presently-disclosed subject matter to determine a measurable characteristic of the peptide biomarkers, and in particular a MS observed mass. As such, “determining an amount” of one or more peptides of Table 1 by MS analysis is inclusive of determining amounts of full-length polypeptides inferred from peptide fragment analysis by MS, specifically-identified peptide fragment amounts, as well as MS observed mass peak analysis.

In another embodiment, a biomarker panel comprises a set of mRNA's and expression of a biomarker is assessed by preparing mRNA/cDNA (i.e. a transcribed polynucleotide) from cells in a patient sample, and by hybridizing the mRNA/cDNA with a reference polynucleotide which is a complement of a biomarker nucleic acid, or a fragment thereof. cDNA can, optionally, be amplified using any of a variety of polymerase chain reaction methods prior to hybridization with the reference polynucleotide. Expression of one or more biomarkers can likewise be detected using quantitative PCR (qPCR) to assess the level of expression of the biomarker(s). Alternatively, any of the many known methods of detecting mutations or variants (e.g. single nucleotide polymorphisms, deletions, etc.) of a biomarker of the invention may be used to detect occurrence of a biomarker in a patient. Preferably, the expression level of one or more senescence biomarkers is normalized to a reference or “housekeeping” gene known in the art, such as tubulin or actin.

In a related embodiment, a mixture of transcribed polynucleotides obtained from the sample is contacted with a substrate having fixed thereto a polynucleotide complementary to or homologous with at least a portion (e.g. at least 7, 10, 15, 20, 25, 30, 40, 50, 100, 500, or more nucleotide residues) of a cellular senescence biomarker nucleic acid. If polynucleotides complementary to or homologous with are differentially detectable on the substrate (e.g. detectable using different chromophores or fluorophores, or fixed to different selected positions), then the levels of expression of a plurality of biomarkers can be assessed simultaneously using a single substrate (e.g. a “gene chip” microarray of polynucleotides fixed at selected positions). When a method of assessing biomarker expression is used which involves hybridization of one nucleic acid with another, it is preferred that the hybridization be performed under stringent hybridization conditions. In a particular embodiment, the level of biomarker mRNA can be determined both by in situ and by in vitro formats in a biological sample using methods known in the art. The term “biological sample” is intended to include tissues, cells, biological fluids and isolates thereof, isolated from a subject, as well as tissues, cells and fluids present within a subject. Many expression detection methods use isolated RNA. For in vitro methods, any RNA isolation technique that does not select against the isolation of mRNA can be utilized for the purification of RNA from tumor cells (see, e.g., Ausubel et at, ed., Current Protocols in Molecular Biology, John Wiley & Sons, New York 1987-1999). Additionally, large numbers of tissue samples can readily be processed using techniques well known to those of skill in the art, such as, for example, the single-step RNA isolation process of Chomczynski (1989, U.S. Pat. No. 4,843,155).

The isolated mRNA can be used in hybridization or amplification assays that include Southern or Northern analyses, polymerase chain reaction analyses and probe arrays. One preferred diagnostic method for the detection of mRNA levels involves contacting the isolated mRNA with a nucleic acid molecule (probe) that can hybridize to the mRNA encoded by the gene being detected. The nucleic acid probe can be, for example, a full-length cDNA, or a portion thereof, such as an oligonucleotide of at least 7, 15, 30, 50, 100, 250 or 500 nucleotides in length and sufficient to specifically hybridize under stringent conditions to a mRNA or genomic DNA encoding a biomarker of the present invention. Other suitable probes for use in the diagnostic assays of the invention are described herein. Hybridization of an mRNA with the probe indicates that the biomarker in question is being expressed.

In one format, the mRNA is immobilized on a solid surface and contacted with a probe, for example by running the isolated mRNA on an agarose gel and transferring the mRNA from the gel to a membrane, such as nitrocellulose. In an alternative format, the probe(s) are immobilized on a solid surface and the mRNA is contacted with the probe(s), for example, in an Affymetrix gene chip array. A skilled artisan can readily adapt known mRNA detection methods for use in detecting the level of mRNA encoded by the biomarkers of the present invention.

An alternative method for determining the level of mRNA biomarker in a sample involves the process of nucleic acid amplification, e.g., by RT-PCR, ligase chain reaction, self sustained sequence replication, transcriptional amplification system, Q-Beta Replicase, rolling circle replication (U.S. Pat. No. 5,854,033) or any other nucleic acid amplification method, followed by the detection of the amplified molecules using techniques well known to those of skill in the art. These detection schemes are especially useful for the detection of nucleic acid molecules if such molecules are present in very low numbers. As used herein, amplification primers are defined as being a pair of nucleic acid molecules that can anneal to 5′ or 3′ regions of a gene (plus and minus strands, respectively, or vice-versa) and contain a short region in between. In general, amplification primers are from about 10 to 30 nucleotides in length and flank a region from about 50 to 200 nucleotides in length. Under appropriate conditions and with appropriate reagents, such primers permit the amplification of a nucleic acid molecule comprising the nucleotide sequence flanked by the primers.

The analysis of a plurality of markers selected from Table 1 (be it at the polypeptide or mRNA level) can be carried out separately or simultaneously with one test sample. Several markers can be combined into one test for efficient processing of a multiple of samples. In addition, one skilled in the art would recognize the value of testing multiple samples (for example, at successive time points) from the same subject. Such testing of serial samples will allow the identification of changes in biomarker levels over time. Increases or decreases in marker levels, as well as the absence of change in marker levels, can provide useful information about the disease status that includes identifying the approximate time from onset of the disease, the presence and amount of functioning tissue, the appropriateness of drug therapies, the effectiveness of various therapies, differentiation of the various types and stages of age-related diseases, identification of the severity of the disease, and identification of the subject's outcome (prognosis), including risk of future events.

A further aspect of the invention relates to a senescent cell detection kit for detecting senescent cells in a sample, the kit comprising means for detecting the presence, in a sample from a test subject, of at least the set of biomarkers according to the invention.

For example, the kit can comprise labeled compounds or agents, each being capable of detecting a biomarker protein or nucleic acid in a biological sample and means for determining the amount of the protein or mRNA in the sample (e.g., an antibody which binds the protein or a fragment thereof, or an oligonucleotide probe which binds to DNA or mRNA encoding the protein). Kits can also include instructions for interpreting the results obtained using the kit.

Provided is a kit comprising means for detecting the presence of at least five, preferably at least six, more preferably at least seven, polypeptides or mRNA's selected from the group consisting of TSPAN13GDNF, C2CD5, CNTLN, FAM214B, PATZ 1, PLXNA3, STAG1, SUSD6, TOLLIP, TRDMT1, ZBTB7A, ARID2, B4GALT7, BCL2L2, CHMP5, CREBBP, DDA1, DYNLT3, EFNB3, ICE1, MEIS1, NOL3, PCIF1, PDLIM4, PDS5B, PLK3, RAI14, RHNO1, SCOC, SLC16A3, SMO, SPIN4, TAF13, TMEM87B, UFM1 and ZNHIT1, or a variant or fragment thereof.

In a specific aspect, a kit comprises means for detecting the presence of at least five polypeptides or mRNA's selected from the group consisting of C2CD5, CNTLN, FAM214B, GDNF, PATZ 1, PLXNA3, STAG1, SUSD6, TOLLIP, TRDMT1 and ZBTB7A, or a variant or fragment thereof. For example, the kit comprises a set of (monoclonal) antibodies, each of which is specifically reactive with a polypeptide of the set comprises at least six, preferably at least seven, more preferably at least eight polypeptides selected from the group consisting of C2CD5, CNTLN, FAM214B, GDNF, PATZ 1, PLXNA3, STAG1, SUSD6, TOLLIP, TRDMT1 and ZBTB7A, or a variant or fragment thereof.

The kit may comprise antibodies reactive with the polypeptides C2CD5, CNTLN, FAM214B, GDNF, PATZ 1, PLXNA3, STAG1, SUSD6, TOLLIP, TRDMT1 and ZBTB7A, or a variant or fragment thereof.

In some embodiments, a kit for the analysis of the biomarker panel is provided that comprises antibodies having specificity for one or more polypeptide biomarkers associated with cell senescence as herein disclosed. The antibodies can be bound to a substrate, as disclosed herein. Such a kit can comprise devices and reagents for the analysis of at least one test sample.

In another embodiment, the kit comprises means for detecting the presence of at least five mRNA's selected from the group consisting of GDNF, C2CD5, CNTLN, FAM214B, PATZ 1, PLXNA3, STAG1, SUSD6, TOLLIP, TRDMT1 and ZBTB7A, or a variant or fragment thereof. For example, the kit comprises a solid substrate having fixed thereto a plurality of oligonucleotides, each being complementary to or homologous with at least a portion (e.g. at least 7, 10, 15, 20, 25, 30, 40, 50, 100, 500, or more nucleotide residues) of a cellular senescence biomarker nucleic acids of the present invention.

For antibody-based kits, the kit can comprise, for example: (1) a first antibody (e.g., attached to a solid support) which binds to a biomarker protein; and, optionally, (2) a second, different antibody which binds to either the protein or the first antibody and is conjugated to a detectable label.

For oligonucleotide-based kits, the kit can comprise, for example: (1) an oligonucleotide, e.g., a detectably labeled oligonucleotide, which hybridizes to a nucleic acid sequence encoding a biomarker protein or (2) a pair of primers useful for amplifying a biomarker nucleic acid molecule. The kit can also comprise, e.g., a buffering agent, a preservative, or a protein stabilizing agent. The kit can further comprise components necessary for detecting the detectable label (e.g., an enzyme or a substrate). The kit can also contain a control sample or a series of control samples which can be assayed and compared to the test sample.

For example, the kit may comprise at least one control or reference sample, preferably the kit comprises a negative control and/or a positive control. The negative control can be any non-senescent cell that does not express any of the upregulated senescent biomarkers according to the invention, or only very low or undetectable concentrations thereof. A positive control may comprise any senescent cell that does express increased levels of one or more of the upregulated senescent biomarkers. Each component of the kit can be enclosed within an individual container and all of the various containers can be within a single package, along with instructions for conducting the analysis and interpreting the results of the assays performed using the kit. Optionally the kits can contain one or more reagents or devices for converting a marker level to a diagnosis or prognosis of the subject.

With the identification of polypeptides that are upregulated in senescent cells, the invention also provides the use of each of them as drug target. Accordingly, the invention provides a drug conjugate for killing a senescent cell, the conjugate comprising a senescent cell targeting moiety configured, in use, to specifically target and bind a senescent cell biomarker selected from the group consisting of TSPAN13, GDNF, FAM214B, PLXNA3, SUSD6, TOLLIP, ZBTB7A, B4GALT7, BCL2L2, CHMP5, DDA1, DYNLT3, NOL3, PDLIM4, PLK3, RAI14, SCOC, SLC16A3, TAF13, TMEM87B, UFM1 and ZNHIT1, and a cytotoxic agent, which kills the bound senescent cell.

In a preferred aspect, the conjugate comprises a senescent cell targeting moiety configured, in use, to specifically target and bind a senescent cell biomarker selected from the group consisting of PLXNA3, SUSD6, FAM214B, GDNF, TOLLIP and ZBTB7A, and a cytotoxic agent, which kills the bound senescent cell.

For example, the targeting moiety is an antibody or an antigen binding fragment thereof, an aptamer, a plastic antibody or a small molecule.

The cytotoxic agent can be a radioisotope, a toxin, toxic peptide or a senolytic drug. In one embodiment, the toxin is selected from the group consisting of doxorubicin, calicheamicin, auristatin, maytansinoid, duocarmycin, camptothecin, anthracyclins, alkaloids, anti-apoptotic inhibitors, lysosome inhibitors, glucose analogues, flavonoids and toxic analogues thereof

In another embodiment, the toxic peptide is Pseudomonas exotoxin A, diphtheria toxin, ricin, gelonin, saporin or pokeweed, or an antiviral protein. In a preferred embodiment, the cytotoxic agent is a senolytic agent, which is toxic against senescent cells, thus avoiding off target effects due to toxicity to non-senescent cells e.g. blood cells. Senolytic agents are known in the art, see for example WO2015/116740A1, herein incorporated by reference. In one embodiment, a drug conjugate of the invention comprises (a) an inhibitor of a Bcl-2 anti-apoptotic protein family member that inhibits at least Bcl-xL; (b) an MDM2 inhibitor; or (c) an Akt specific inhibitor.

As is demonstrated in Example 3 herein below, it was surprisingly found that the mRNA levels of TSPAN13 are increased in different types of senescent cells. Flow cytometry was used to sort senescent cellular populations on the basis of a high or low cell surface expression of TSPAN13. Interestingly, TSPAN13 positive cells were found to show increased levels of the known senescence markers p16 and p21. TSPAN13 expressed on the plasma membrane of senescent cells can be targeted by antibody-drug conjugates (ADCs).

Accordingly, in a preferred aspect, the conjugate comprises a senescent cell targeting moiety which, when in use, specifically targets and binds to TSPAN13, and which is conjugated to a cytotoxic agent. In one embodiment, the invention provides a recombinant monoclonal antibody, preferably against TSPAN13, that is covalently bound to a cytoxic agent (also called warheads) via synthetic cleavable or non-cleavable linkers. In a specific embodiment, the drug conjugate is an antibody against TSPAN13 that is conjugated to an inhibitor of one or more BCL-2 anti-apoptotic protein family members wherein the inhibitor inhibits at least Bcl-xL and is selected from ABT-263, ABT-737, WEHI-539, and A-l 155463. For example, the YSPAN13 antibody is conjugated to ABT-263 (also known as Navitoclax) or ABT-737.

In another aspect, the conjugate comprising a senescent cell targeting moiety, like an antibody, which specifically targets and binds to GDNF, which targeting moiety is conjugated to a cytotoxic agent.

In a preferred aspect, the conjugate comprising a senescent cell targeting moiety, like an antibody, which specifically targets and binds to PLXNA3, which targeting moiety is conjugated to a cytotoxic agent.

In another preferred aspect, the conjugate comprising a senescent cell targeting moiety, like an antibody, which specifically targets and binds to SUSD6, which targeting moiety is conjugated to a cytotoxic agent.

In another aspect, the conjugate comprising a senescent cell targeting moiety, like an antibody, which specifically targets and binds to TOLLIP, which targeting moiety is conjugated to a cytotoxic agent.

In another aspect, the conjugate comprising a senescent cell targeting moiety, like an antibody, which specifically targets and binds to ZBTB7A, which targeting moiety is conjugated to a cytotoxic agent.

In a preferred embodiment, the conjugate comprising a senescent cell targeting moiety, like an antibody, which specifically targets and binds to TSPAN13, which targeting moiety is conjugated to a cytotoxic agent.

The term “antibody” broadly encompasses naturally occurring forms of antibodies and recombinant antibodies such as single-chain antibodies, chimeric and humanized antibodies and multi-specific antibodies as well as fragments and derivatives of all of the foregoing, which fragments and derivatives have at least an antigenic binding site. Antibody derivatives may comprise a protein or chemical moiety conjugated to the antibody.

The term “antibody” is used in the broadest sense and covers fully assembled antibodies, antibody fragments that can bind antigen (e.g., Fab′, F′(ab)2, Fv, single chain antibodies, diabodies), and recombinant peptides comprising the foregoing.

In one embodiment, the target moiety is a monoclonal antibody, which as used herein refers to an antibody obtained from a population of substantially homogeneous antibodies, i.e., the individual antibodies comprising the population are identical except for possible naturally-occurring mutations that may be present in minor amounts.

“Antibody fragments” comprise a portion of an intact antibody, preferably the antigen-binding or variable region of the intact antibody. Examples of antibody fragments include Fab, Fab′, F(ab′)2, and Fv fragments; diabodies; linear antibodies; single-chain antibody molecules; and multispecific antibodies formed from antibody fragments. Papain digestion of antibodies produces two identical antigen-binding fragments, called “Fab” fragments, each with a single antigen-binding site, and a residual “Fc” fragment, whose name reflects its ability to crystallize 35 readily. Pepsin treatment yields an F(ab′)2 fragment that has two antigen-combining sites and is still capable of cross-linking antigen.

“Fv” is the minimum antibody fragment that contains a complete antigen recognition and binding site. In a two-chain Fv species, this region consists of a dimer of one heavy- and one light-chain variable domain in tight, non-covalent association. In a single-chain Fv species, one heavy- and one light-chain variable domain can be covalently linked by flexible peptide linker such that the light and heavy chains can associate in a “dimeric” structure analogous to that in a two-chain Fv species. It is in this configuration that the three CDRs of each variable domain interact to define an antigen-binding site on the surface of the VH-VL dimer. Collectively, the six CDRs confer antigen-binding specificity to the antibody. However, even a single variable domain (or half of an Fv comprising only three CDRs specific for an antigen) has the ability to recognize and bind antigen, although at a lower affinity than the entire binding site.

The Fab fragment also contains the constant domain of the light chain and the first constant domain (CH1) of the heavy chain. Fab fragments differ from Fab′ fragments by the addition of a few residues at the carboxy terminus of the heavy-chain CH1 domain including one or more cysteines from the antibody hinge region. Fab′-SH is the designation herein for Fab′ in which the cysteine residue(s) of the constant domains bear a free thiol group. F(ab′)2 antibody fragments originally were produced as pairs of Fab′ fragments that have hinge cysteines between them.

Antibodies against a (human) senescent cell polypeptide target, e.g. TSPAN13, for use in a drug conjugate as herein provided can be obtained from commercial suppliers or can be manufactured by methods known in the art.

For example, polyclonal antibodies can be prepared by immunizing a suitable subject (e.g., chicken, rabbit, goat, mouse, or other mammal) with a senescent biomarker protein immunogen. The antibody titer in the immunized subject can be monitored over time by standard techniques, such as with an ELISA using immobilized biomarker protein. At an appropriate time after immunization, e.g., when the antibody titers are highest, antibody-producing cells can be obtained from the subject and used to prepare monoclonal antibodies by standard techniques, such as the hybridoma technique originally described by Kohler and Milstein (1975) Nature 256:495-497, the human B cell hybridoma technique (Kozbor et al. (1983) Immunol. Today 4:72), the EBV-hybridoma technique (Cole et al. (1985) in Monoclonal Antibodies and Cancer Therapy, ed. Reisfeld and Sell (Alan R. Liss, Inc., New York, N.Y.), pp. 77-96) or trioma techniques. The technology for producing hybridomas is well known in the art.

Alternative to preparing monoclonal antibody-secreting hybridomas, a monoclonal antibody can be identified and isolated by screening a recombinant combinatorial immunoglobulin library (e.g., an antibody phage display library) with a biomarker protein to thereby isolate immunoglobulin library members that bind the biomarker protein. Kits for generating and screening phage display libraries are commercially available (e.g., the Pharmacia Recombinant Phage Antibody System, Catalog No. 27-9400-01; and the Stratagene SurfZAP θ Phage Display Kit, Catalog No. 240612).

The invention also provides a pharmaceutical composition comprising a drug conjugate according to the invention, and a pharmaceutically acceptable vehicle. The composition may target more than one drug targets of the invention. For example, it comprises two or more drug conjugates selected from the group of conjugates that specifically target and bind to a senescent cell biomarker selected from the group consisting of TSPAN13, PLXNA3, SUSD6, FAM214B, GDNF, TOLLIP and ZBTB7A, and a cytotoxic agent, which kills the bound senescent cell. Preferably, the pharmaceutical composition comprises at least a drug conjugate comprising a TSPAN13 targeting moiety, like an antibody, which targeting moiety is conjugated to a cytotoxic agent.

Also provided is a drug conjugate according to the invention for use as a medicament. For example, the drug conjugate is suitably used in treating, postponing, preventing or ameliorating an age-related disease.

An age-related pathology may include any disease or condition which is fully or partially mediated by the induction or maintenance of a non-proliferating or senescent state in a cell or a population of cells in a subject. Examples include age-related tissue or organ decline which may lack visible indication of pathology, or overt pathology such as a degenerative disease or a function-decreasing disorder. For example, Alzheimer's disease, Parkinson's disease, cataracts, macular degeneration, glaucoma, atherosclerosis, acute coronary syndrome, myocardial infarction, stroke, hypertension, idiopathic pulmonary fibrosis (IPF), chronic obstructive pulmonary disease (COPD), osteoarthritis, type 2 diabetes, obesity, fat dysfunction, coronary artery disease, cerebrovascular disease, periodontal disease, and cancer treatment-related disability such as atrophy and fibrosis in various tissues, brain and heart injury, and therapy-related myelodysplastic syndromes. Additionally, an age-related pathology may include an accelerated aging disease such as Hutchinson-Gilford progeria syndrome, Werner syndrome, Cockayne syndrome, xeroderma pigmentosum, ataxia telangiectasia, Fanconi anemia, dyskeratosis congenital, aplastic anemia, idiopathic pulmonary fibrosis, and others.

Preferred age-related diseases include atherosclerosis, cardiovascular disease, cancer, arthritis, glaucoma, cataracts, osteoporosis, type 2 diabetes, hypertension, Alzheimer's disease or other type of dementia.

In a further embodiment, the invention provides a method for treating a senescence-associated pathology comprising administering to a subject in need thereof a pharmaceutical composition comprising a therapeutically-effective amount of a drug conjugate according to the invention that selectively kills senescent cells over non-senescent cells.

By selectively killing one or more senescent cells is meant a composition of the invention that does not appreciably kill non-senescent cells at the same concentration. Accordingly, the median lethal dose or LD50 of the composition in non-senescent cells may be about 2 to about 50 times higher than the LD50 of the composition in senescent cells. As used herein, the LD50 is the concentration of composition required to kill half the cells in the cell sample. For example, the LD50 of the composition in non-senescent cells may be greater than about 2, about 3, about 4, about 5, about 6, about 7, about 8, about 9 or about 10 times higher than the LD50 of the composition in senescent cells. Alternatively, the LD50 of the composition in non-senescent cells may be greater than about 10, about 15, about 20, about 25, about 30, about 35, about 40, about 45, or about 50 times higher than the LD50 of the composition in senescent cells. Additionally, the LD50 of the composition in non-senescent cells may be greater than 50 times higher than the LD50 of the composition in senescent cells. In certain embodiments, the LD50 of the composition in non-senescent cells is about 2 to about 10 times higher than the LD50 of the composition in senescent cells. In an exemplary embodiment, the LD50 of the composition in non-senescent cells is about 3 to about 6 times higher than the LD50 of the composition in senescent cells.

A senescence-associated pathology may include any disease or condition which is fully or partially mediated by the induction or maintenance of a non-proliferating or senescent state in a cell or a population of cells in a subject. Non-limiting examples include cardiovascular diseases such as angina, aortic aneurysm, arrhythmia, brain aneurysm, cardiac diastolic dysfunction, cardiac fibrosis, cardiac stress resistance, cardiomyopathy, carotid artery disease, coronary thrombosis, endocarditis, hypercholesterolemia, hyperlipidemia, mitral valve prolapsed, and peripheral vascular disease; inflammatory or autoimmune diseases such as herniated intervertebral disc, inflammatory bowel disease, kyphosis, oral mucositis, lupus, interstitial cystitis, scleroderma, and alopecia; neurodegenerative diseases such as dementia, Huntington's disease, motor neuron dysfunction, age-related memory decline, and depression/mood disorders; metabolic diseases such as diabetic ulcer and metabolic syndrome; pulmonary diseases such as age-related loss of pulmonary function, asthma, bronchiectasis, cystic fibrosis, emphysema, and age-associated sleep apnea; gastrointestinal diseases such as Barrett's esophagus; age-related disorders such as liver fibrosis, muscle fatigue, oral submucosa fibrosis, pancreatic fibrosis, benign prostatic hyperplasia (BPH), and age-related sleep disorders; reproductive disorders such as menopause (male and female), egg supply (female), sperm viability (male), fertility (male and female), sex drive, and erectile function and arousal (male and female); dermatological diseases such as atopic dermatitis, cutaneous lupus, cutaneous lymphomas, dysesthesia, eczema, eczematous eruptions, eosinophilic dermatosis, fibrohistocytic proliferations of skin, hyperpigmentation, immunobullous dermatosis, nevi, pemphigoid, pemphigus, pruritis, psoriasis, rashes, reactive neutrophilic dermatosis, rhytides, and urticarial; and other diseases such as diabetic wound healing, post-transplant kidney fibrosis, and carotid thrombosis.

For therapeutic applications, a therapeutically effective amount of a drug conjugate of the invention is administered to a subject. A “therapeutically effective amount” is an amount of the therapeutic composition sufficient to produce a measurable response (e.g., cell death of senescent cells, an anti-aging response, an improvement in symptoms associated with a degenerative disease, or an improvement in symptoms associated with a function-decreasing disorder). Actual dosage levels of active ingredients in a therapeutic composition can be varied so as to administer an amount of the active compound(s) that is effective to achieve the desired therapeutic response for a particular subject. The selected dosage level will depend upon a variety of factors including the activity of the therapeutic composition, formulation, the route of administration, combination with other drugs or treatments, age, the age-related disease or condition, the degenerative disease, the function-decreasing disorder, the symptoms, and the physical condition and prior medical history of the subject being treated. In some embodiments, a minimal dose is administered, and dose is escalated in the absence of dose-limiting toxicity. Determination and adjustment of a therapeutically effective dose, as well as evaluation of when and how to make such adjustments, are known to those of ordinary skill in the art of medicine.

The frequency of dosing may be daily or once, twice, three times or more per week or per month, as needed as to effectively treat the symptoms. The timing of administration of the treatment relative to the disease itself and duration of treatment will be determined by the circumstances surrounding the case. Treatment could begin immediately, such as at the site of the injury as administered by emergency medical personnel. Treatment could begin in a hospital or clinic itself, or at a later time after discharge from the hospital or after being seen in an outpatient clinic. Duration of treatment could range from a single dose administered on a one-time basis to a life-long course of therapeutic treatments. Typical dosage levels can be determined and optimized using standard clinical techniques and will be dependent on the mode of administration.

A subject may be a rodent, a human, a livestock animal, a companion animal, or a zoological animal. In one embodiment, the subject may be a rodent, e.g. a mouse, a rat, a guinea pig, etc. In another embodiment, the subject may be a livestock animal. Non-limiting examples of suitable livestock animals may include pigs, cows, horses, goats, sheep, llamas and alpacas. In still another embodiment, the subject may be a companion animal. Non-limiting examples of companion animals may include pets such as dogs, cats, rabbits, and birds. In yet another embodiment, the subject may be a zoological animal. As used herein, a “zoological animal” refers to an animal that may be found in a zoo. Such animals may include non-human primates, large cats, wolves, and bears. In a preferred embodiment, the subject is a human.

The human subject may be of any age. However, since senescent cells are normally associated with aging, a human subject may be an older human subject. In some embodiments, the human subject may be about 30, 35, 40, 45, 50, 55, 60, 65, 70, 75, 80, 85, 90, 95 years of age or older. In some preferred embodiments, the human subject is 30 years of age or older. In other preferred embodiments, the human subject is 40 years of age or older. In other preferred embodiments, the human subject is 45 years of age or older. In yet other preferred embodiments, the human subject is 50 years of age or older. In still other preferred embodiments, the human subject is 55 years of age or older. In other preferred embodiments, the human subject is 60 years of age or older. In yet other preferred embodiments, the human subject is 65 years of age or older. In still other preferred embodiments, the human subject is 70 years of age or older. In other preferred embodiments, the human subject is 75 years of age or older. In still other preferred embodiments, the human subject is 80 years of age or older. In yet other preferred embodiments, the human subject is 85 years of age or older. In still other preferred embodiments, the human subject is 90 years of age or older.

### LEGEND TO THE FIGURES

FIG. 1. Meta-analysis of senescent fibroblast transcriptomics. Experimental Design. Seven RNA-seq datasets, including three types of senescence and six different strains of fibroblasts, were used to build a stimulus-specific signature and a general signature of senescent fibroblasts irrespective of the stimulus. Each signature was built using three methods: a negative binomial generalized linear model (GLM), the Fisher- and the Inverse Normal-p-value combination. Only genes with a p-value<=0.01 calculated by the three methods and with expression unchanged or in the opposite direction in quiescence were included in each signature. The signature for Ionizing radiation-induced senescence (IRIS) was built with the negative binomial GLM only because only one dataset was available.

The number of genes comprising each signature is displayed: 1721 genes for the Replicative Senescence (RS) signature, 1586 genes for the Oncogene-Induced Senescence (OIS) signature, 2688 genes for the Ionizing Radiation-Induced Senescence (IRIS) signature and 726 genes for the senescence signature of fibroblasts irrespective of the stimulus

FIG. 2. Characteristics of the core senescence-associated signature. A. Experimental Design. RNA-seq datasets obtained from the indicated studies of melanocytes, keratinocytes and astrocytes were compared to the senescence signature for fibroblasts. The intersection of genes differentially expressed (p-value<=0.01) in all the datasets are shown in the flower plot. B. Heatmap of the 37 genes of the core signature of senescence. The figure shows the logarithm base 2 of the fold change for each cell type with respect to proliferating cells. C. Gene Ontology (GO) terms enriched in the core senescence signature. The plot shows the enriched GO terms in the up- (red) and down- (blue) regulated genes of the signature. Bars indicate the logarithm base 10 of the p-value. D. Pathways enriched in the core signature of senescence. The pathways enriched in genes within the core senescence signature (B) are listed with their corresponding p-value and source.

FIG. 3. Temporal dynamics of the senescence transcriptome. A. Experimental Design. Fibroblasts (HCA-2, yellow), melanocytes (red) and keratinocytes (magenta) were exposed to ionizing radiation (IR) and RNA harvested 4, 10 or 20 days later.

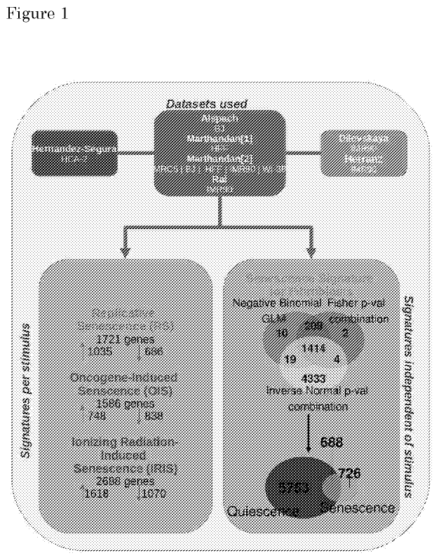

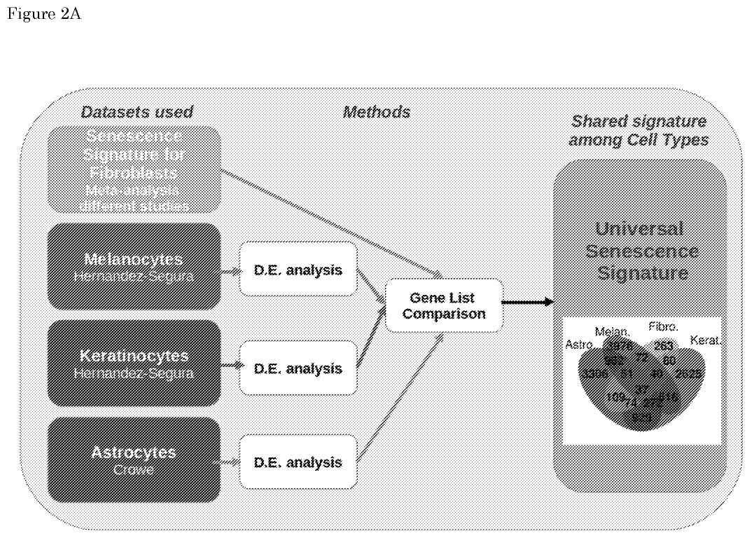

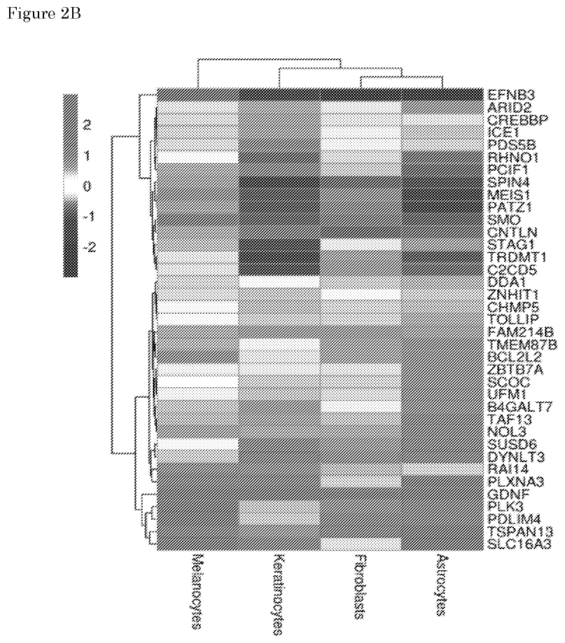

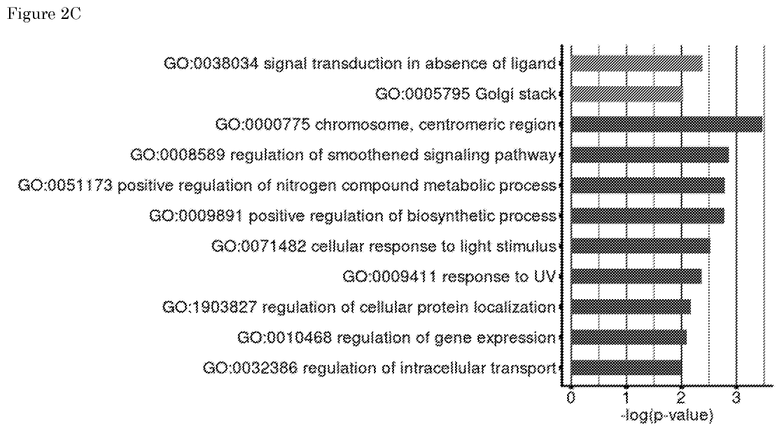

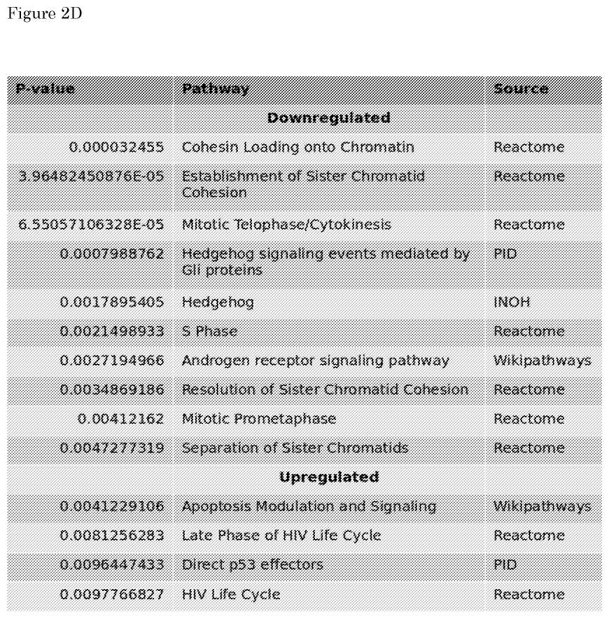

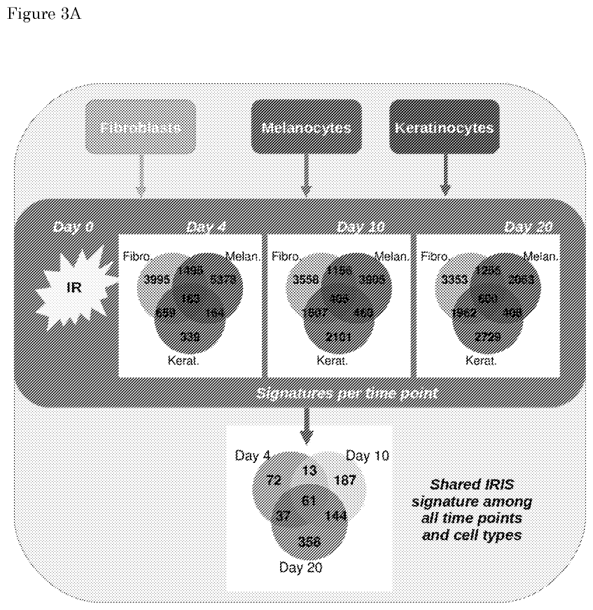

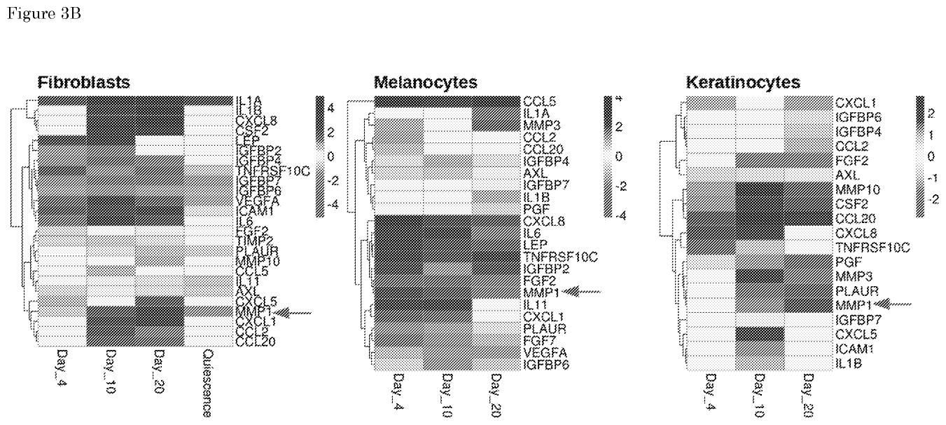

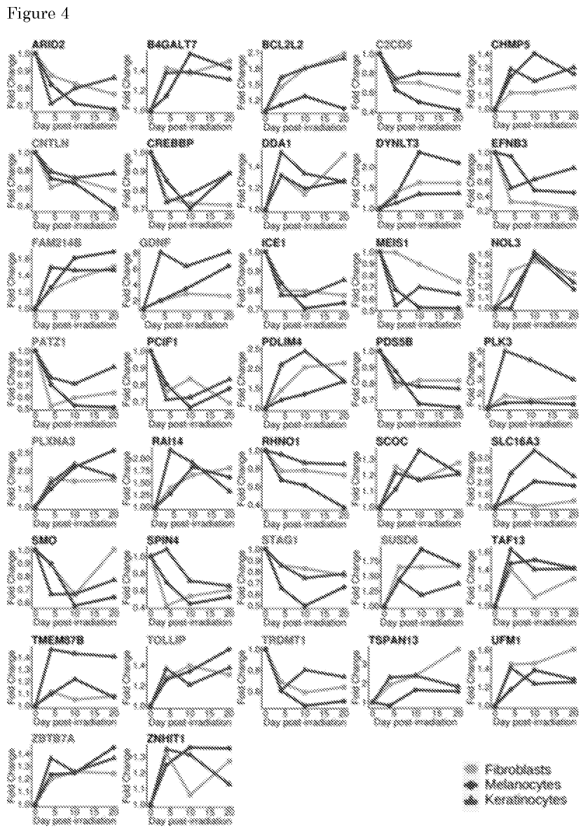

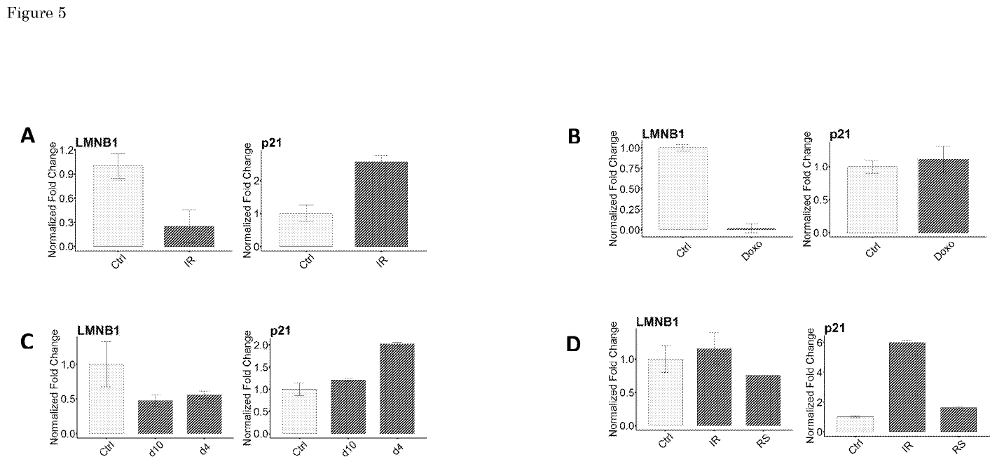

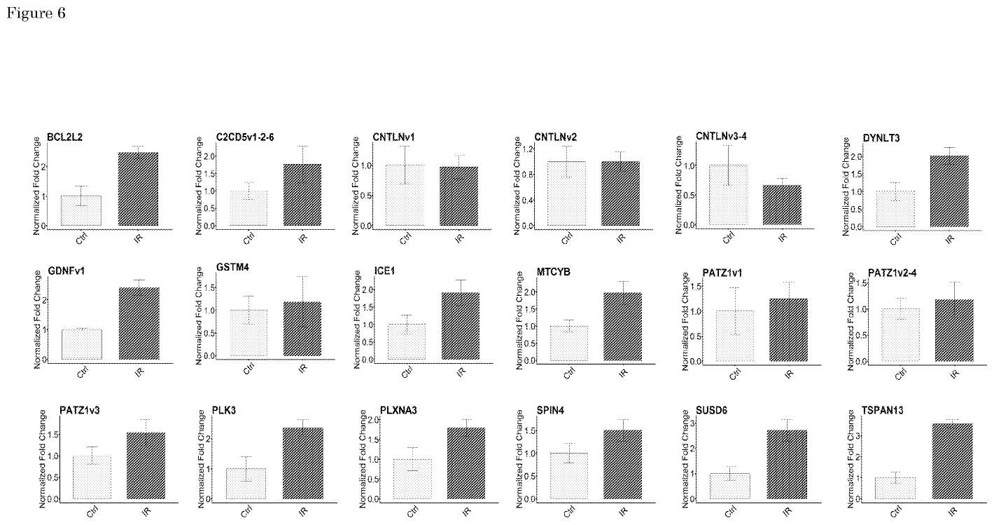

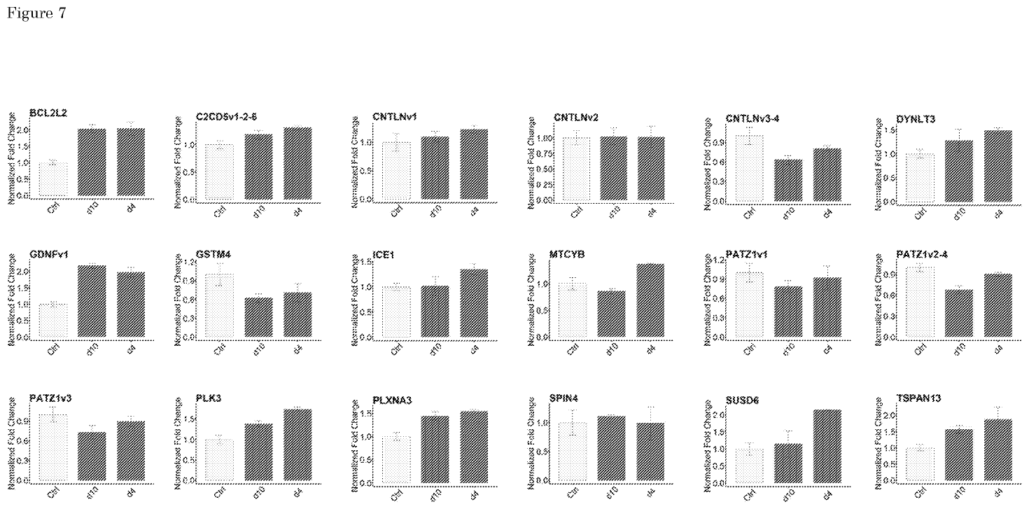

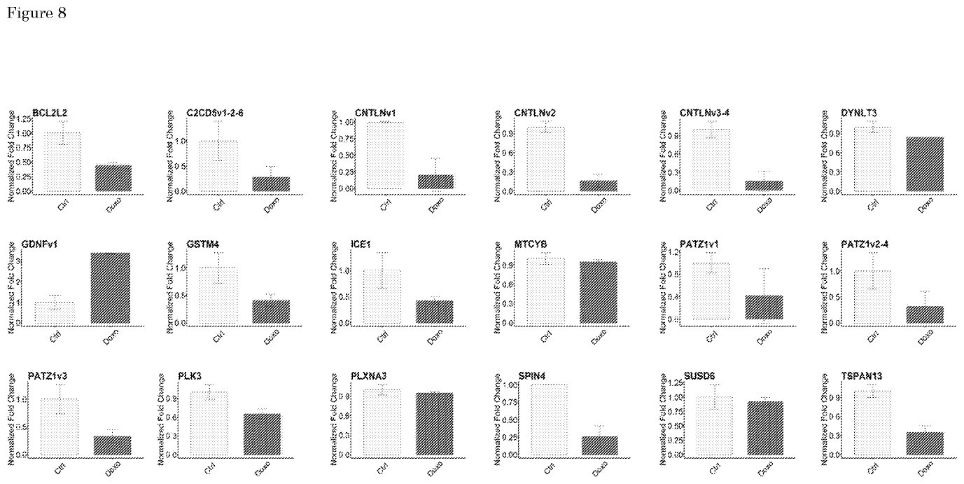

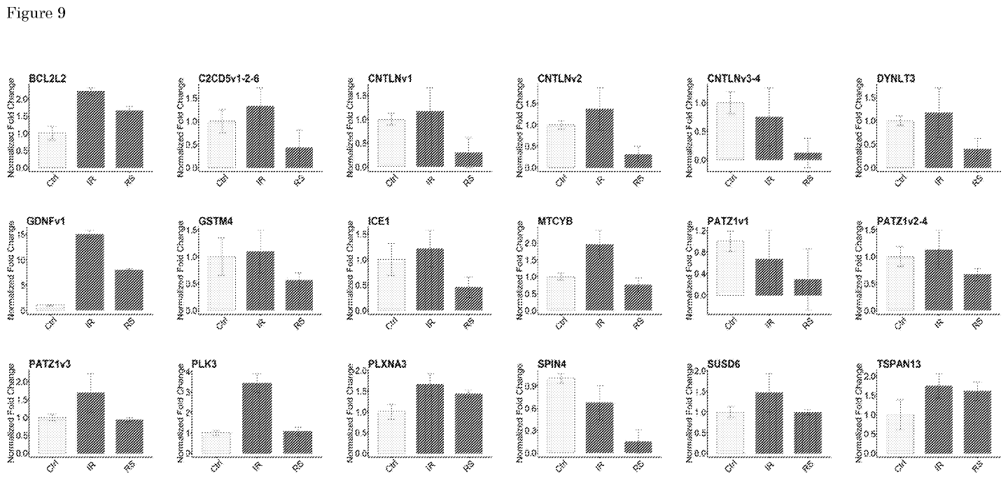

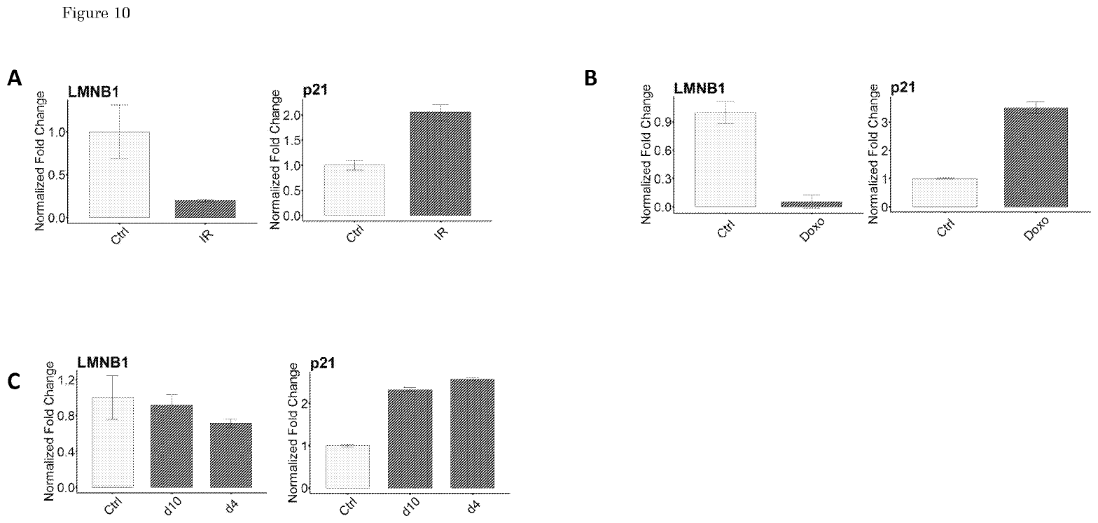

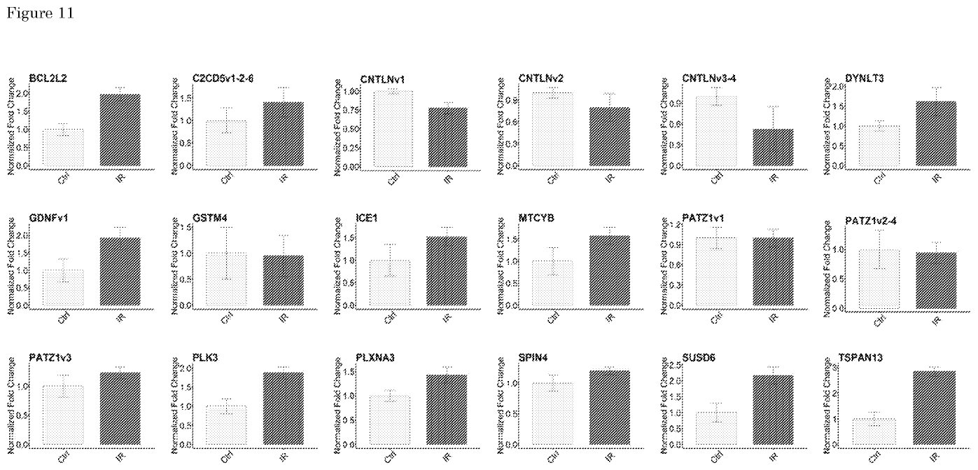

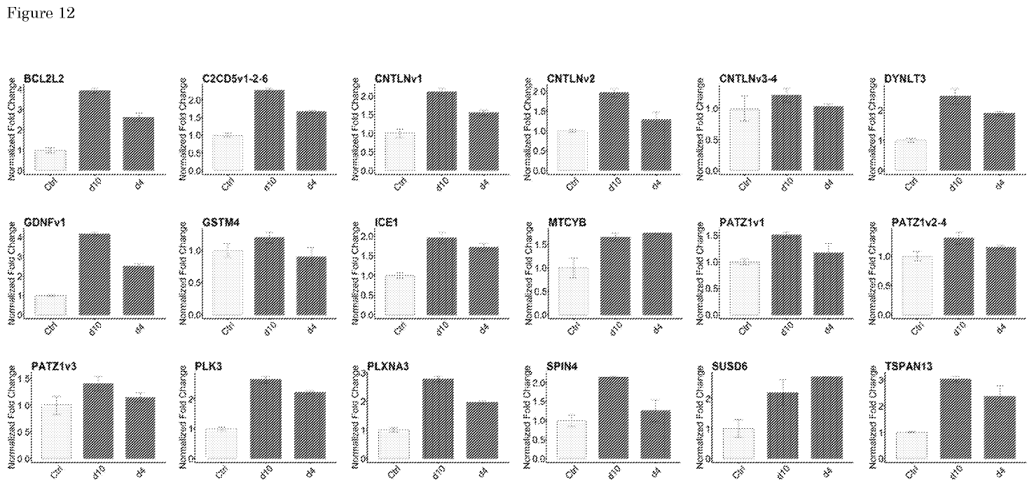

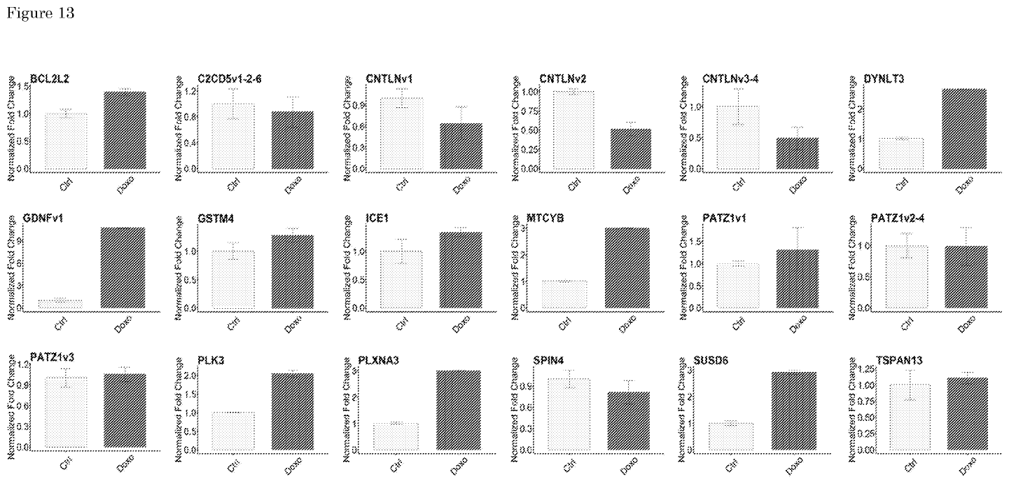

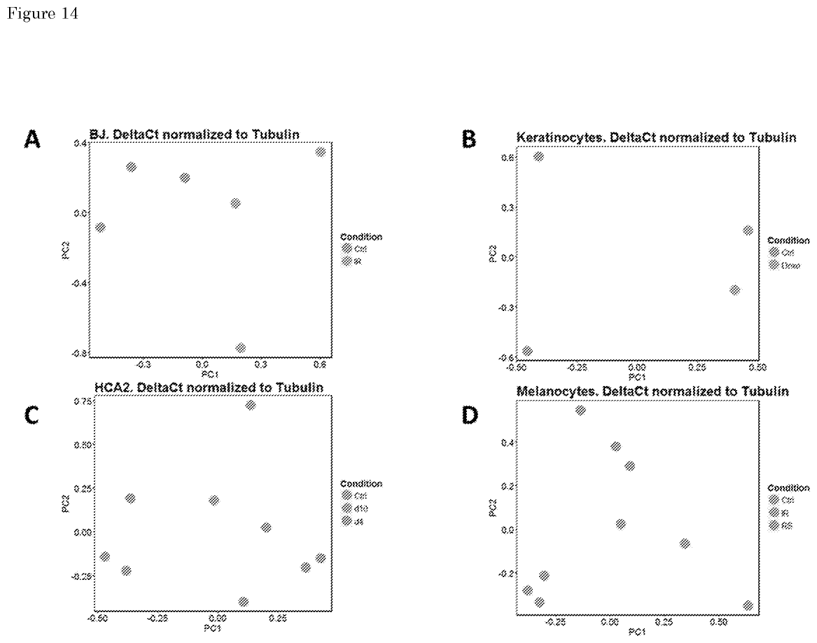

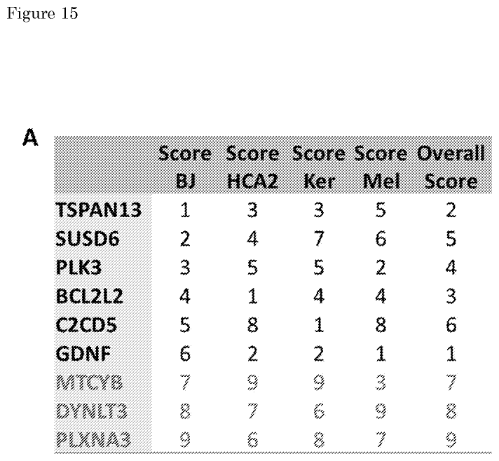

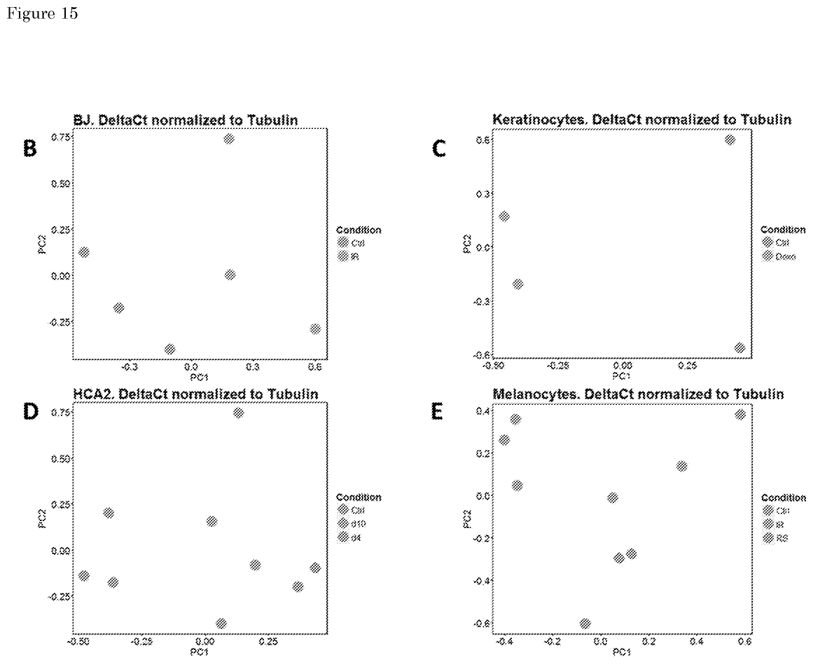

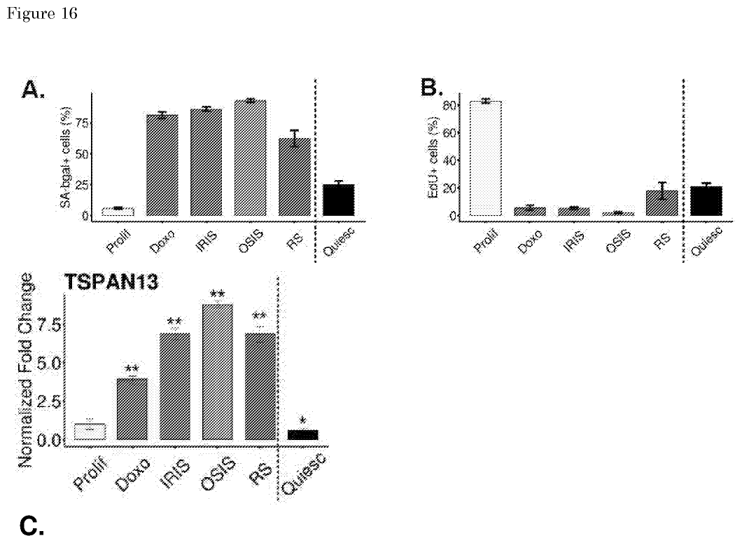

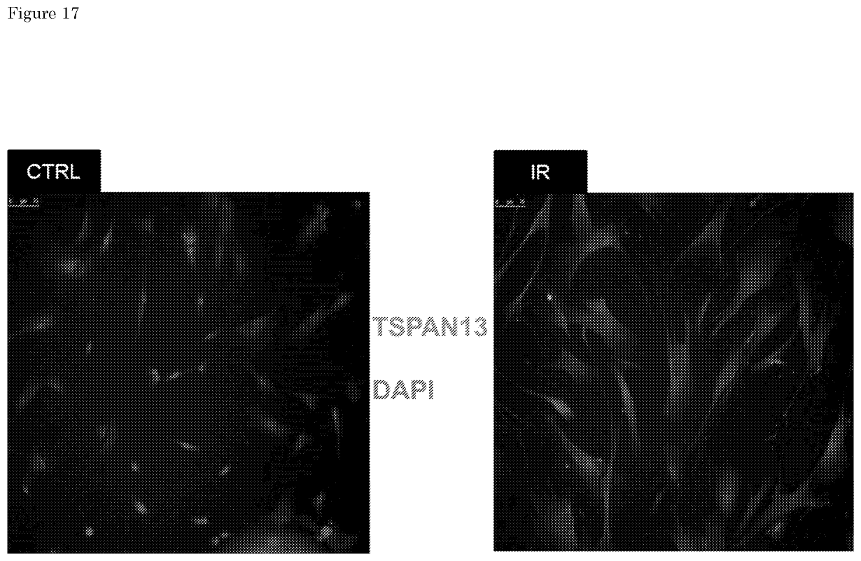

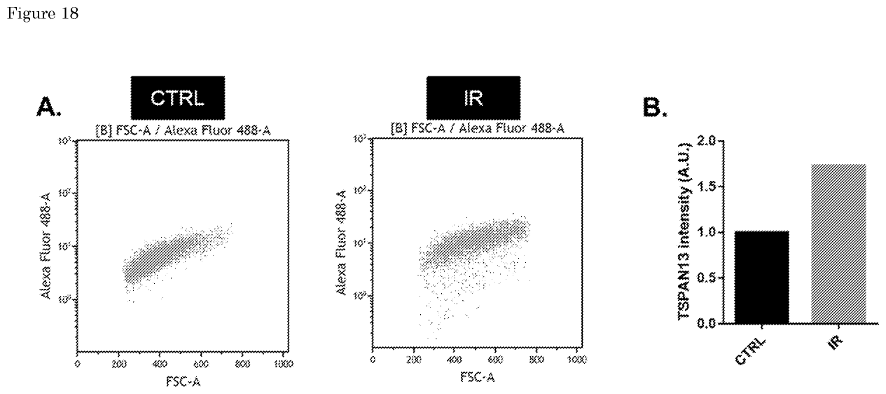

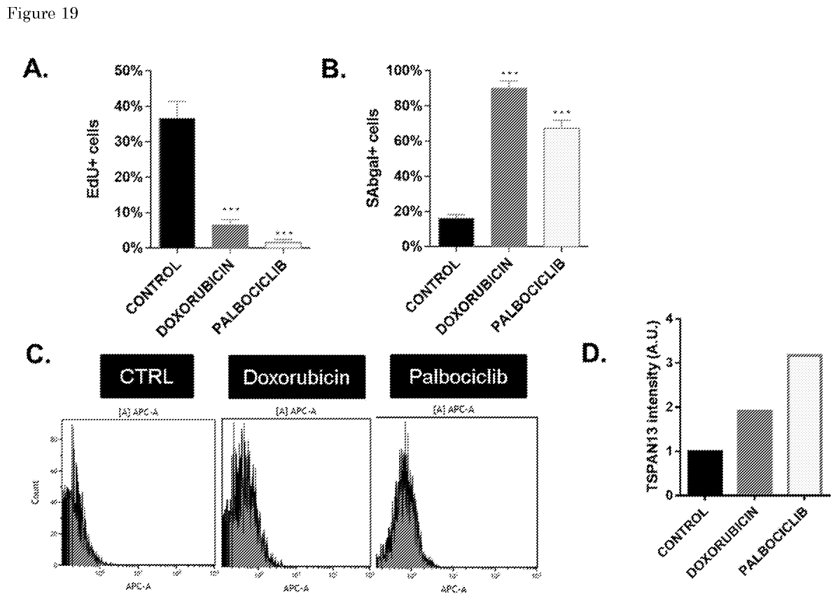

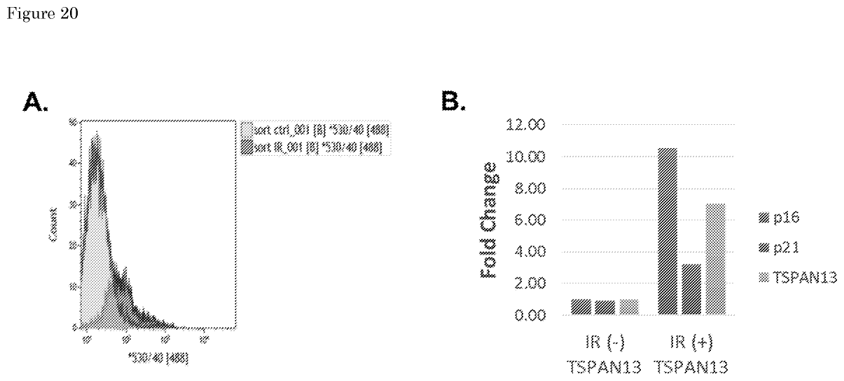

Transcriptomes of the different cell types and intervals after senescence induction were obtained by RNA-seq. A time-point signature with genes differentially expressed (p-value<=0.01) in all three cell types and a shared IR-induced Senescence (IRIS) signature with genes shared by all cell types and time points (p-value<=0.01) were generated. B. Heatmap showing dynamics of the SASP for each cell type. Known SASP factors that were significantly differentially expressed at least one time point in each cell type are shown. The heatmap shows the logarithm base 2 of the fold change for each time post-irradiation with respect to proliferating cells. Quiescence was measured only on fibroblasts. The violet arrows highlight MMP1, the only SASP factor commonly regulated at day 10 and 20 in all cell types.

FIG. 4. Dynamic changes in expression of genes in the core senescence signature. Each panel shows one of the 37 genes in the core signature of senescence at the indicated points before and after irradiation. All genes show a dynamic temporal behavior at the time points tested: day 0 (proliferation), day 4, day 10 and day 20 post-irradiation. Notably, all genes show a similar trend in the three cell types tested: fibroblasts (yellow), melanocytes (red) and keratinocytes (magenta). Genes in red correspond to those that reached significance (p-value<=0.01) at all time points tested.

FIG. 5. Gene expression of senescence markers normalized to Tubulin. Senescence was confirmed through SA-bgal (data not shown) in all samples and at least one established marker of senescence: downregulation of LMNB1 or upregulation of p21. Tubulin was used as a reference gene to calculate deltaCt values. A) LMNB1 and p21 expression in proliferating (Ctrl) and irradiated (IR) BJ fibroblasts. B) LMNB1 and p21 expression in proliferating (Ctrl) and doxorubicin-treated (Doxo) keratinocytes. C) LMNB1 and p21 expression in proliferating (Ctrl) and either 4 or 10 days post-irradiation (d4 and d10, respectively) of HCA2 fibroblasts. D) LMNB1 and p21 expression in proliferating (Ctrl), irradiated (IR) and Replicative Senescent (RS) melanocytes.

FIG. 6. Gene expression of pre-selected genes of the Senescence Signature normalized to Tubulin in BJ fibroblasts. Gene expression of different biomarker genes within the Senescence Signature of the invention was measured by real time-PCR in Proliferating (Ctrl) and irradiated (IR, day 10 post-irradiation) BJ fibroblasts. Delta-Ct values were calculated using tubulin according to the method developed by Livak et al (2001. Methods 25(4)). Each condition includes three biological replicates, each run in technical duplicates. Error bars show the standard error of the mean. Of note, results were not always statistically significant (data not shown).

FIG. 7. Gene expression of pre-selected biomarkers of the Senescence Signature normalized to Tubulin in HCA2 fibroblasts. Gene expression of different biomarker genes within the Senescence Signature was measured by real time-PCR in Proliferating (Ctrl) and either 4 or 10 days post-irradiation (d4 and d10, respectively) HCA2 fibroblasts. Delta-Ct values were calculated using tubulin according to the method developed by Livak et al (2001. Methods 25(4)). Each condition includes three biological replicates, each run in technical duplicates. Error bars show the standard error of the mean. Of note, results were not always statistically significant (data not shown).

FIG. 8. Gene expression of pre-selected biomarkers of the Senescence Signature normalized to Tubulin in Keratinocytes. Gene expression of different biomarker genes within the Senescence Signature was measured by real time-PCR in Proliferating (Ctrl) and doxorubicin-treated (Doxo) keratinocytes. Delta-Ct values were calculated using tubulin according to the method developed by Livak et al (2001. Methods 25(4)). Each condition includes two biological replicates, each run in technical duplicates. Error bars show the standard error of the mean. Of note, results were not always statistically significant (data not shown).

FIG. 9. Gene expression of pre-selected biomarkers of the Senescence Signature normalized to Tubulin in Melanocytes. Gene expression of different biomarker genes within the Senescence Signature was measured by real time-PCR in Proliferating (Ctrl), irradiated (IR) or Replicative Senescent (RS) melanocytes. Delta-Ct values were calculated using tubulin according to the method developed by Livak et al (2001. Methods 25(4)). Each condition includes three biological replicates, each run in technical duplicates. Errorbars show the standard error of the mean. Of note, results were not always statistically significant (data not shown).

FIG. 10. Gene expression of senescence markers normalized to Actin. Senescence was confirmed through SA-bgal (data not shown) in all samples and at least another marker of senescence: downregulation of LMNB1 or upregulation of p21. Actin was used as a reference gene to calculate deltaCt values. A) LMNB1 and p21 expression in proliferating (Ctrl) and irradiated (IR) BJ fibroblasts. B) LMNB1 and p21 expression in proliferating (Ctrl) and doxorubicin-treated (Doxo) keratinocytes. C) LMNB1 and p21 expression in proliferating (Ctrl) and either 4 or 10 days post-irradiation (d4 and d10, respectively) of HCA2 fibroblasts.

FIG. 11. Gene expression of pre-selected genes of the Senescence Signature normalized to Actin in BJ fibroblasts. Gene expression of different genes within the Senescence Signature was measured by real time-PCR in Proliferating (Ctrl) and irradiated (IR, clay 10 post-irradiation) BJ fibroblasts.

FIG. 12. Gene expression of pre-selected genes of the Senescence Signature normalized to Actin in HCA2 fibroblasts. Gene expression of different genes within the Senescence Signature was measured by real time-PCR in Proliferating (Ctrl) and either 4 or 10 days post-irradiation (d4 and d10, respectively) HCA2 fibroblasts.

FIG. 13. Gene expression of pre-selected genes of the Senescence Signature normalized to Actin in Keratinocytes. Gene expression of different genes within the Senescence Signature was measured by real time-PCR in Proliferating (Ctrl) and doxorubicin-treated (Doxo) keratinocytes.

FIG. 14. Principal Component Analysis (PCA) of deltaCt values of pre-selected genes are able to discriminate senescent from proliferating cells. These genes included: BCL2L2, C2CD5 (primer pair amplifying variants 1, 2 and 6), DYNLT3, GDNF (primer pair amplifying variant 1), MTCYB, PLK3, PLXNA3, SUSD6 and TSPAN13. A) PCA plot of Proliferating (Ctrl) and irradiated (IR) BJ fibroblasts using selected genes. B) PCA plot of Proliferating (Ctrl) and doxorubicin-treated (Doxo) keratinocytes using selected genes. C) PCA plot of Proliferating (Ctrl) and day 4 and 10 post-irradiation (d4 and d10, respectively) HCA2 fibroblasts using selected genes. D) PCA plot of Proliferating (Ctrl), irradiated (IR) and Replicative Senescent (RS) melanocytes using selected genes.

FIG. 15. A panel of only 6 biomarkers (“minimal core signature”) is necessary and sufficient to discriminate Senescent from Proliferating cells in different cell types. A) Contribution of each biomarker gene to the sample separation on the Principal Component 1 (X-axis) on FIG. 11 was calculated for each set of samples. The biomarker with the higher contribution was scored as “1” and that with the lowest contribution was scored as “9” for each of the cell types (BJ=BJ fibroblasts, HCA2=HCA2 fibroblasts, Ker=keratinocytes, Mel=melanocytes). An overall score for each biomarker was calculated, being “1” the biomarker that had a higher contribution in all the samples and “9” the biomarker that had the lowest contribution in all samples. Panels B) through E) show new PCA plots built using 6 final genes: GDNF (primer pair amplifying variant 1), TSPAN13, BCL2L2, PLK3, SUSD6 and C2CD5 (primer pair amplifying variant 1, 2 and 6) for each sample. B) PCA plot of core transcriptional markers of senescence for proliferating (Ctrl) versus Irradiated (IR) BJ fibroblasts. C) PCA plot of core transcriptional markers of senescence for proliferating (Ctrl) versus doxorubicin-treated (Doxo) keratinocytes. D) PCA plot of core transcriptional markers of senescence for proliferating (Ctrl) versus day 4 or day 10 post-irradiation (d4 and d10, respectively) HCA2 fibroblasts. E) PCA plot of core transcriptional markers of senescence for proliferating (Ctrl), Irradiated (IR) and replicative Senescent (RS) melanocytes.

FIG. 16. TSPAN13 mRNA expression is up-regulated in senescent fibroblasts. Human skin fibroblasts BJ were induced to senescence by doxorubicin (Doxo), ionizing radiation (IRIS), hydrogen peroxide (OSIS) or replicative exhaustion (RS). Proliferating (Prolif) or quiescent (Quiesc) cells were used as control. Panel A: percentage of cells with activation of the Senescence-Associated beta-galactosidase (SA-bgal) enzyme. Panel B: percentage of cells with active DNA synthesis (EdU, reporter for proliferation). Panel C: levels of TSPAN13 mRNA measured by qPCR after normalization of the tubulin mRNA levels. N=3 independent experiments. *p<=0.05, **p<=0.01.

FIG. 17. TSPAN13 protein expression is up-regulated in senescent fibroblasts. Human skin fibroblasts BJ were induced to senescence by ionizing radiation (IR). 9 days later, control (CTRL) or IR cells were stained with an antibody against TSPAN13 (green) and counterstained with DAPI to reveal nuclei (blue).

FIG. 18. TSPAN13 protein expression is up-regulated in senescent fibroblasts. Human skin fibroblasts BJ were induced to undergo senescence by ionizing radiation (IR). 9 days later, control (CTRL) or IR cells were stained with an antibody against TSPAN13 and signal intensity measured via flow cytometer. Panel A: dot-plots of fluorescence intensity of the 2 populations. Panel B: quantification of the data in Panel A.

FIG. 19. TSPAN13 protein expression is up-regulated in senescent fibroblasts. Human skin fibroblasts WI38 were induced to senescence by Doxorubicin or Palbociclib, and the induction of senescence (compared to control cells) confirmed by the percentage of cells with activation of the Senescence-Associated beta-galactosidase enzyme (SA-bgal, panel A) is reported and by the percentage of cells with active DNA synthesis (EdU, panel B) is shown. Cells were stained with an antibody against TSPAN13 and signal intensity measured via flow cytometer. Panel C: dot-plot of fluorescence intensity of the 3 populations. Panel D: quantification.

FIG. 20. TSPAN13-positive cells show an increased expression level of other senescence markers. Human skin fibroblasts BJ were induced to senescence by ionizing radiation (IR), stained with an antibody against TSPAN13 and the signal intensity measured via flow cytometer. The plot in panel (A) shows a shift in TSPAN13 expression in IR cells (red bell) compared to control cells (green bell). Panel (B) shows IR cells with high (IR+ TSPAN13) or low expression (IR− TSPAN13) of TSPAN13 that were sorted in 2 independent tubes and RNA isolated. TSPAN13-high expressing cells show high level of other senescence markers p16 and p21 measured by qPCR and normalized to Tubulin when compared to TSPAN13-low expressing cells.

### EXPERIMENTAL SECTION

### Materials and Methods

### Cell Strains and Culture

Human foreskin fibroblasts HCA2 were obtained from the laboratory of O. Pereira-Smith (University of Texas Health Science Center, San Antonio); human foreskin fibroblasts BJ were purchased from ATCC (Cat: CRL-2522); MEFs were produced from 13.5 day embryos as previously described (Demaria 2010); mouse primary skin microvascular endothelial cells were purchased from Cellbiologics (Cat: C57-6064).

All cells were cultured in 5% oxygen for at least 4 doublings prior to use. Fibroblasts were cultured in DMEM (Thermo Fisher Scientific) enriched with 10% fetal bovine serum (FBS,

GE Healthcare Life Sciences) and 1% penicillin/streptomycin (Lonza). Endothelial cells were grown in endothelial cell growth media (ATCC). Quiescence was induced by culturing cells for 48 hours in DMEM supplemented with 0.2% FBS. For replicative senescence, cells were passaged (re-cultured at 30-40% density until they reached 70-80% confluency) until proliferation ceased (˜65 population doublings for BJ cells). For oxidative stress-induced senescence, cells were treated with 200 uM hydrogen peroxide (Sigma Aldrich) for 2 h, followed by drug removal and culturing in fresh DMEM supplemented with 10% FBS. Treatment was repeated at days 0, 3 and 6, with the media refreshed every 2 days, and cells harvested on day 10 after the first treatment. Doxorubicin (Tebu-bio) was used at 250 nM for 24 h. The medium was replaced by DMEM supplemented with 10% FBS and refreshed every 2 days. Cells were harvested on day 7 after treatment. For irradiation-induced senescence, cells exposed to 10 Gy γ-radiation using a 137Cesium source and medium was refreshed every 2 days.

Cells were harvested at day 10 after irradiation for most experiments and validations. For the time series, cells were harvested at day 4, 10 and 20 after irradiation.

### SA-Beta Galactosidase Assay

Cells were plated in 24-well plates, fixed in glutaraldehyde/formaldehyde (2%/2%) for 10-15 min and stained overnight with an X-Gal solution using a commercial kit (Biovision). Cells were counter-stained with 1 μg/ml 4′,6-diamidino-2-phenylindole (DAPI, Sigma-Aldrich, D9542) for 20 min. Images were acquired at 100× magnification, and the number of cells counted by the software ImageJ (www.rsbweb.nih.gov/ij/). Positive cells were scored manually. Samples were done in triplicate, counting at least 100 cells for each replicate.

### EdU Staining

Cells were cultured for 24 h in the presence of EdU, and fixed and stained using a commercial kit (Click-iT EdU Alexa Fluor 488 Imaging kit; Thermo Fisher Scientific). Images were acquired at 400× magnification, and quantified using ImageJ (www.rsbweb.nih.gov/ij/). Samples were done in triplicate, counting at least 100 cells for each replicate.

### Real Time-PCR

Total RNA was prepared using the Isolate II Rna Mini Kit (Bioline). 255-500 ng of RNA was reverse transcribed using a kit (Applied Biosystems). qRT-PCR reactions were performed as described [43] using the Universal Probe Library system (Roche) and a SENSIFast Probe kit (Bioline). Tubulin was used for normalization of CT values. All samples were run with a technical replicate, using 2-3 biological replicates. An unpaired two-tailed Student's t-test was used to determine statistical significance based on delta-CT values. P values of 0.05 or less were considered significant.

### RNAseq

Cells were prepared for RNA extraction using an RNAeasy mini kit (Invitrogen). Samples were treated with Qiasol lysis buffer and total RNA was isolated using a Qiacube robot as per the manufacturer's instructions (Invitrogen). The RNA was quantified using a NanoDrop and RNA quality was measured using a BioAnalyzer chip (Agilent). Purified RNA samples were sent to the University of Minnesota BioMedical Genomics Center for library preparation (polyA-enrichment) and Illumina HiSeq RNA sequencing as per the manufacturer's protocols (Illumina). Size selection for 50 bp paired end sequencing was carried out for insert sizes of ˜200 bp, and sequencing was performed at ≥220 M reads per lane of the HiSeq2500 flow cell. Average Quality scores for the completed run across all samples was >30, with an average of number of reads for each pooled sample >10 million reads. The raw data have been deposited in the ArrayExpress database (https://www.ebi.ac.uk/arrayexpress/; access number: E-MTAB-5403).

### Public Datasets

A summary of the public datasets and samples used are in tables S1 and S4. The raw data from the public datasets was obtained from the “GEO repository”. Six public datasets of the transcriptomes of senescent fibroblasts were included: 1) Alspach et al, 2014 [16] (“GSE56293”) used RS of BJ cells to study SASP induction; 2) Dikovskaya et al, 2015 [17] (“GSE70668”) used IMR90 cells to study multinucleation in OIS (induced by Ras), and used cells synchronized in mitosis; 3) Herranz et al, 2015 [18] (“GSE61130”) studied the SASP in OIS (induced by Ras) in IMR90 cells; 4) Marthandan et al, 2015 [20] (“GSE63577”) used MRC-5 and HFF cells to study the effect of rotenone at different population doubling levels, and we used only the first (proliferation) and last time points for HFF cells; 5) Marthandan et al, 2016 [19] (“GSE64553”) used five fibroblast strains (BJ, WI-38, IMR90, HFF and MRC-5) to study RS; 6) Rai et al, 2014 [21] (“GSE53356”), used IMR90 cells to study the chromatin landscape of RS. One public dataset produced by Crowe et al, 2016 [30] (“GSE58910”) studying OSIS in astrocytes was used for the core signature of senescence shared by different cell types.

### Quality Control and Alignment of Transcriptome Datasets

Raw data was downloaded as fastq files using the SRA Toolkit 2.6.2. Quality control of all samples, including our own, was performed using FastQC software v0.11.5 and low quality reads (Average Quality: <20) were discarded. End-trimming was performed when necessary using Trimmommatic 0.36. Samples were aligned to the GRCh38 genome using STAR-2.5.1b aligner and a raw read count table was directly obtained from STAR output. Only genes annotated as protein-coding were included in the analysis.

### Meta-Analysis of Fibroblasts

Heterogeneity of the data was evaluated with a PCA-plot of the log-transformed normalized counts for the protein-coding genes. For meta-analyses of specific stimuli and of the fibroblast senescence signature, we used three methods: a negative-binomial generalized linear model (GLM), the Fisher p-value combination and the Inverse Normal p-value combination. The first approach used the R-package DESeq2 for differential expression analysis using senescence versus proliferation as the main variable. In cases using more than one cell type, cell type was included as a covariate.

The other two approaches used the R-package MetaRNAseq. First, differential expression analysis using the DESeq2 package was performed for each dataset and p-values were combined by two methods: Fisher and Inverse Normal. Genes with a multiple-testing adjusted (using Benjamini-Hochberg procedure) p-value<=0.01 in the negative-binomial GLM and a combined p-value<=0.01 in the other two methods were included in the corresponding signature. Genes that were also differentially regulated in quiescence samples (adjusted p-value<=0.01 and sign of the fold change in the same direction as senescence) were removed as possible senescence markers after the meta-analysis was finished. Enriched pathways and gene onthology terms in the differentially expressed genes in the fibroblast senescence signature were evaluated using the online tool “Over-representation analysis” of the Consensus Path DB-human (http://cpdb.molgen.mpg.de/).

### Core Senescence Signature Shared by Different Cell Types

Differential expression analysis was also performed with DESeq2 for each dataset separately and lists of differentially expressed genes were compared to the senescence signature of fibroblasts without combining p-values. Only genes with multiple-testing adjusted p-value<=0.01 in every dataset and the fibroblast signature (negative binomial GLM method) were included in the core senescence signature.

### Plots

All plots were made using the following R-packages: “pheatmap”, “ggplot2”, “ggfortify”, “RColorBrewer” and “VennDiagram”.

### Example 2: Minimal Core Signature to Identify Senescent Cells

This example describes the analysis of gene expression of different biomarker genes within the Senescence Signature in order to obtain a “minimal core senescence signature” comprising a panel of biomarkers that is necessary and sufficient to discriminate senescent cells from non-senescent cells.

To that end, a set of pre-selected biomarkers identified in Example 1 was measured by real time-PCR in 4 different cell types undergoing senescence:
HCA2 fibroblasts: Control vs day 4 and day 10 post-irradiation
BJ fibroblasts: Control vs Irradiated
Melanocytes: Control vs Irradiated and replicative senescence (in triplicates)
Keratinocytes: Control vs Doxorubicin-treated (in duplicates)

### Materials and Methods

Cell Strain and Culture.

Human foreskin fibroblasts BJ, human neonatal melanocytes and human neonatal keratinocytes were purchased from ATCC (Cat: CRL-2522, PCS-200-012 and PCS-200-010, respectively). BJ fibroblasts were cultured in DMEM medium (Thermo Fisher Scientific) enriched with 10% fetal bovine serum (FBS, GE Healthcare Life Sciences) and 1% penicillin/streptomycin (Lonza). Keratinocytes were cultured in CnT-Prime, Epithelial. Culture Medium (CellnTec, CnT-PR) without addition of antibiotics. Melanocytes were cultured in RPMI medium enriched with 10% fetal bovine serum and 1% penicillin/streptomycin and supplemented with 200 nM 12-O-Tetradecanoylphorbol 13-acetate (TPA, Sigma-Aldrich), 200 nM cholera toxin (Sigma-Aldrich), 10 nM endothelin 1 (Sigma-Aldrich) and 10 ng/ml human stem cell factor (Peprotech). All cells were cultured in 5% oxygen, 5% CO₂ and 37° C. and tested regularly for mycoplasma infection.

Sample Preparation.

For ionizing radiation-induced senescence (IRIS), cells were subjected to a 10 Gy dose of γ-radiation using a ¹³⁷Cesium source and medium was refreshed every 2 days. Cells were harvested at day 4 and/or day 10 after irradiation. For replicative senescence (RS), cells were propagated in culture for ˜4 months (re-cultured at 30-40% density every time they reached 70-80% confluence) until they stopped growing (˜PD 65). Doxorubicin (Tebu-bio) was used in a concentration of 250 nM for 24 hours. Cells were washed once with their corresponding medium and then new medium was added and refreshed every 2 days. Cells were harvested on day 7 after treatment. Proliferating controls for each condition were generated stimulating cells with the same PD of the treated samples or treated with vehicle (PBS) in the case of doxorubicin.

Confirmation of Senescence by SA-βGal Assay.

Cells were plated in a 24-well plate, fixed in a mixture of glutaraldehyde and formaldehyde (2%/2%) for 3-5 minutes and stained overnight with an X-Gal solution using a commercial kit (Biovision). Cells were counter-stained with a 1 μg/ml 4′,6-diamidino-2-phenylindole (DAPI, Sigma-Aldrich, D9542) solution for 20 min. Images were acquired at 100× magnification, and the number of cells counted by the software ImageJ (www.rsbweb.nih.gov/ij/). The number of positive cells was counted manually. The data for SA-bgal staining is not shown.

Real Time-PCR.

Total RNA was prepared using the Isolate II Rna

Mini Kit (Bioline). 100-500 ng of RNA was reverse transcribed into cDNA using a kit (Applied Biosystems). qRT-PCR reactions were performed as using the Universal Probe Library system (Roche) and a SENSIFast Probe kit (Bioline) according to manufacturer's instructions. Expression of tubulin or actin was used to normalize the expression of CT values. Samples were run with a technical replicate and in 2-3 biological replicates. An unpaired two-tailed Student's t-test was used to determine statistical significance based on delta-CT values.

Principal Component Analysis (PCA):

Genes to be used for the principal component analysis were pre-selected based on the reproducibility of the changes in expression between proliferating and senescent cells in the qPCR results. Genes that followed the same trend as predicted by the original analysis (based on RNAseq results) in most of the samples were used to build a PCA plot. These genes included: BCL2L2, C2CD5 (primer pair amplifying variants 1, 2 and 6), DYNLT3, GDNF (primer pair amplifying variant 1), MTCYB, PLK3, PLXNA3, SUSD6 and TSPAN13. Contribution of each gene to the sample separation on the Principal Component 1 (X-axis) was calculated for each set of samples. Gene with the higher contribution was scored as “1” and gene with the lowest contribution was scored as “9”. A list for all the samples used in the analysis was built and an overall score for each gene on all the samples was calculated, being “1” the gene that had a higher contribution in all the samples and “9” the gene that had the lowest contribution in all samples. Finally, the three genes that scored last (7, 8 and 9) were discarded. A new PCA plot was build using 6 final genes: GDNF (primer pair amplifying variant 1), TSPAN13, BCL2L2, PLK3, SUSD6 and C2CD5 (primer pair amplifying variant 1, 2 and 6).

### List of Primers Used:

| | Di- | Sequence | UPL |
|---|---|---|---|
| Gene | rection | (5′→3′) | probe |
| Tub | FW | cttcgtctccgccatcag | 40 |
| | RV | cgtgttccaggcagtagagc | |
| ActB | FW | ctaaggccaaccgtgaaaag | 64 |
| | RV | accagaggcatacagggaca | |
| P21 | FW | tcactgtcttgtacccttgtgc | 32 |
| | RV | ggcgtttggagtggtagaaa | |
| LMNB1 | FW | gtgctgcgagcaggagac | 3 |
| | RV | ccattaagatcagattccttcttagc | |
| BCL2L2 | FW | tggatggtggcctacctg | 28 |
| | RV | cgtccccgtatagagctgtg | |
| C2CD5v1- | FW | tccttttcaccttccaagtcc | 35 |
| 2-6 | RV | gcactaccacttcccattcc | |
| CNTLNv1 | FW | aaggcagcaatacaagaattgaa | 53 |
| | RV | ttgacctcatcatcttcaccag | |
| CNTLNv2 | FW | gaaggatatggatattaccctggtc | 17 |
| | RV | tgctgggctgtatgtgtactg | |
| CNTLNv3- | FW | gaatttgtatggtctttgtggaaa | 80 |
| 4 | RV | tcgttttggtggtacacatctt | |
| DYNLT3 | FW | gtgctctaccggcgtgtc | 25 |
| | RV | cagcattgaagccaacctc | |
| GDNFv1 | FW | atgtccaacctagggtctgc | 70 |
| | RV | catcccataacttcatcttaaagtcc | |
| GSTM4 | FW | tgctacagccctgactttga | 60 |
| | RV | tgatcttgtctccaacaaaccat | |
| ICE1 | FW | tgggaatacatatttgccattg | 74 |
| | RV | atgcgtccagatccatttct | |
| MTCYB | FW | caacaaccgctatgtatttcgt | 83 |
| | RV | ggtttttatgtactacaggtggtcaa | |
| PATZ1v1 | FW | gcaacttctgcagtatctgtaacc | 13 |
| | RV | accgtggtgggttttaacat | |
| PATZ1v2- | FW | agccttacatctgccagagc | 80 |
| 4 | RV | gcttgatatgtccgttcaagtg | |
| PATZ1v3 | FW | tgcagtatctgtaaccgaggtc | 32 |
| | RV | gtgagcatttctggccttct | |
| PLK3 | FW | gaaggtgggggattttgg | 6 |
| | RV | gggtgccacagatggtct | |
| PLXNA3 | FW | gagggcactctggctctg | 17 |
| | RV | cagaagttgccgttgatctg | |
| SPIN4 | FW | actatttccctacagcagaacagg | 80 |
| | RV | gcttgcccacgagactgt | |
| SUSD6 | FW | ctgcagattcagagaacagtgac | 82 |
| | RV | ctcatgcttctttcaacagtgg | |
| TSPAN13 | FW | tcaacctgctttacaccttgg | 84 |
| | RV | aatcagcccgaagccaat | |

### Materials:

| REAGENT or RESOURCE | SOURCE | IDENTIFIE |
|---|---|---|
| Chemicals, Peptides, and Recombinant Proteins | | |
| DMEM, GlutaMAX Supplement, pyruvate | Gibco | Cat: 31966021 |
| CnT-Prime, Epithelial Culture Medium | CellnTec | Cat: CnT-PR |
| RPMI 1640 Medium, GlutaMAX Supplement | Thermo Fisher Scientific | Cat: 61870044 |
| 12-O-Tetradecanoylphorbol 13-acetate (TPA) | Sigma-Aldrich | 16561-29-8 |
| Cholera toxin | Sigma-Aldrich | C8052 |
| Endothelin 1 | Sigma-Aldrich | 117399-94-7 |
| Human stem cell factor | Peprotech | 300-07 |
| Fetal Bovine Serum | GE Healthcare Bio-Sciences | Cat: SV30160.03 |
| Pen/strep stock, 10,000/10,000, 100 ml | Lonza | Cat: LODE17-602E |
| Doxorubicin hydrochloride | Tebu-bio | Cat: BIA-D1202-1 |
| 25% Glutaraldehyde solution | Thermo Fisher Scientific | Cat: MERC8.20603.1000 |
| 16% Formaldehyde (w/v), Methanol-free | Thermo Fisher Scientific | Cat: 28906 |
| Diamidino-2-phenylindole (DAPI) | Sigma-Aldrich | Cat: D9542 |
| Commercial Assays | | |
| Beta-Galactosidase Staining Kit | BioVision | |
| Isolate II RNA Mini Kit | Bioline | Cat: BIO-52073 |
| High-Capacity cDNA Reverse Transcription kit | Applied Biosystems | Cat: 4368813 |
| Universal Proble Library System | Roche | Cat: 04683633001 |
| SENSIFast Probe no-ROX One-step Kit | Bioline | Cat: BIO-76001 |
| Experimental Models: Cell Lines | | |
| BJ fibroblasts | ATCC | Cat: CRL-2522 |
| Human Neonatal Keratinocytes | ATCC | Cat: PCS-200-010 |
| Human Neonatal Melanocytes | ATCC | Cat: PCS-200-012 |
| Software and Algorithms | | |
| R and associated R-packages (pheatmap, ggplot2, | Bioconductor, Cran R project | |
| ggfortify, RcolorBrewer, VennDiagram) | | |
| ImageJ | https://imagej.nih.gov/ij/download.html | |

### Results

FIG. 6 confirms senescence of each cell sample under investigation by analysis of at least an established marker of senescence: downregulation of LMNB1 or upregulation of p21 normalized to Tubulin.

FIGS. 7-9 show gene expression of pre-selected biomarker genes of the Senescence Signature normalized to Tubulin in BJ fibroblasts (FIG. 7), HCA2 fibroblasts (FIG. 8) and keratinocytes (FIG. 9) as measured by real time-PCR. Delta-Ct values were calculated using tubulin according to the method developed by Livak et al (2001. Methods 25(4)). Each condition includes three biological replicates, each run in technical duplicates. Error bars show the standard error of the mean. Of note, results were not always statistically significant (data not shown).

FIG. 10 confirms senescence of each cell sample under investigation by analysis of at least an established marker of senescence: downregulation of LMNB1 or upregulation of p21 normalized to Actin.

FIGS. 11-13 show gene expression of pre-selected biomarker genes of the Senescence Signature normalized to Actin in BJ fibroblasts (FIG. 11), HCA2 fibroblasts (FIG. 12) and keratinocytes (FIG. 13) as measured by real time-PCR. Delta-Ct values were calculated using tubulin according to the method developed by Livak et al (2001. Methods 25(4)). Each condition includes three biological replicates, each run in technical duplicates. Error bars show the standard error of the mean. Of note, results were not always statistically significant (data not shown).

Thereafter, biomarker genes to be used for the principal component analysis were pre-selected based on the reproducibility of the changes in expression between proliferating and senescent cells in the qPCR results. Genes that followed the same trend (up- or down-regulation regardless the statistical significance) as predicted by the original analysis (based on RNAseq results) in most of the samples were used to build a PCA plot of the deltaCt values normalized to Tubulin. These biomarkers included BCL2L2, C2CD5 (primer pair amplifying variants 1, 2 and 6), DYNLT3, GDNF (primer pair amplifying variant 1), MTCYB, PLK3, PLXNA3, SUSD6 and TSPAN13. See FIG. 14, showing the PCA plots for BJ fibroblasts, keratinocytes, HCA fibroblasts and melanocytes.

Finally, a set of only 6 biomarker genes (minimal core signature) was identified by analyzing the contribution of each gene to the sample separation on the Principal Component 1 (X-axis) on FIG. 14 was calculated for each set of samples. The biomarker gene with the highest contribution was scored as “1” and gene with the lowest contribution was scored as “9” for each of the cell types (BJ=BJ fibroblasts, HCA2=HCA2 fibroblasts, Ker=keratinocytes, Mel=melanocytes). An overall score for each gene was calculated, being “1” the gene that had a higher contribution in all the samples and “9” the gene that had the lowest contribution in all samples. The three genes that scored last (in red) were discarded. This resulted in a set comprising the biomarkers TSPAN13, GDNF, C2CD5, SUSD6, BCL2L2 and PLK3 (see FIG. 15).

### Example 3: TSPAN13 Expression is Increased on Senescent Cells

TSPAN13 is a cell surface protein whose function is poorly characterized. This example demonstrates the association of TSPAN13 with cellular senescence.

FIG. 16 shows that the mRNA levels of TSPAN13 are increased in different types of senescent cells. In FIG. 17, we show that the TSPAN13 protein is more expressed by using immuno-fluorescence. Using flow cytometric and additional cells/stimuli analysis, FIGS. 18 and 19 indicate similar TSPAN13 upregulation. In FIG. 20, flow cytometry is used to sort senescent cellular populations on the basis of a high or low expression of TSPAN13. Interestingly, increased TSPAN13 levels were found to correlate with increased levels of other senescence markers p16 and p21.

Based on its cell surface localization, TSPAN13 is an ideal for drug targeting. For example, a drug conjugate for killing a senescent cell can be configured which comprises (i) a TSPAN13 targeting agent that, when in use, specifically targets and binds to TSPAN13 and (ii) a cytotoxic agent, which kills the bound senescent cell.

### Experimental Conditions

### Gene Expression TSPAN13 (FIG. 16)

Human foreskin fibroblasts BJ were purchased from ATCC (Cat: CRL-2522). Cells were cultured in 5% oxygen, 5% CO₂ and 37 C and using DMEM medium (Thermo Fisher Scientific) enriched with 10% fetal bovine serum (FBS, GE Healthcare Life Sciences) and 1% penicillin/streptomycin (Lonza). Cells were tested regularly for mycoplasma infection.

### Sample Preparation.

Quiescence was induced by culturing the cells for 48 hours in DMEM supplemented with 0.2% FBS. For ionizing radiation-induced senescence (IRIS), cells were subjected to a 10 Gy dose of γ-radiation using a ¹³⁷Cesium source and medium was refreshed every 2 days. Cells were harvested at day 10 after irradiation. For replicative senescence (RS), cells were propagated in culture for ˜4 months (re-cultured at 30-40% density every time they reached 70-80% confluence) until they stopped growing (˜PD 65). For oxidative stress-induced senescence (OSIS), cells were treated with 200 μM of hydrogen peroxide (Sigma Aldrich) for 2 hours, followed by drug removal and culturing in fresh DMEM supplemented with 10% FBS. Treatment was repeated at day 0, 3 and 6, with medium refreshed every 2 days in between, and cells harvested on day 10 after the first treatment.

Doxorubicin (Tebu-bio) was used in a concentration of 250 nM for 24 hours. The medium was then replaced by normal DMEM supplemented with 10% FBS and refreshed every 2 days. Cells were harvested on day 7 after treatment.

Proliferating controls for each condition were generated stimulating cells with the corresponding vehicles and/or considering the same PD of the treated samples. When only one control for multiple conditions is shown, it represents the average of controls for each condition.

### SA-βGal Assay.

Cells were plated in a 24-well plate, fixed in a mixture of gluteraldehyde and formaldehyde (2%/2%) for 10-15 minutes and stained overnight with an X-Gal solution using a commercial kit (Biovision). Cells were counter-stained with a 1 μg/ml 4′,6-diamidino-2-phenylindole (DAPI, Sigma-Aldrich, D9542) solution for 20 min. Images were acquired at 100× magnification, and the number of cells counted by the software ImageJ (www.rsbweb.nih.gov/ij/). The number of positive cells was counted manually.

### EdU Staining.

Cells were cultured for 24 hours in the presence of EdU, and fixed and stained using a commercial kit (Click-iT EdU Alexa Fluor 488 Imaging kit; Thermo Fisher Scientific). Images were acquired at 400× magnification, quantified using ImageJ (www.rsbweb.nih.gov/ij/). In all cases, both for SA-6 gal assay and for EdU staining, samples were done in triplicates and at least 100 cells were counted in each replicate and corresponding barplots were generated, where error bars represent the Standard Error of the Mean (SEM).

Real Time-PCR Reverse transcriptase PCR was performed using the High-Capacity cDNA Reverse Transcription Kit (Applied Biosystems, cat #4368813). qPCR for TSPAN13 was performed using a LC480 (Roche) and SensiFAST Probe Lo-ROX Kit (Bioline, cat #84020). Tubulin was used as a reference gene. Primers: TSPAN13, F-CCCTCAACCTGCTTTACACC, R-AATCAGCCCGAAGCCAAT, UPL probe #84; Tubulin, F-CTTCGTCTCCGCCATCAG, R-CGTGTTCCAGGCAGTAGAGC, UPL probe #40. An unpaired two-tailed Student's t-test was used to determine statistical significance based on delta-CT values. P values of 0.05 or less were considered statistically significant.

### Immunofluorescence (IF) Analysis of TSPAN13 Expression (FIG. 17)

9d IR and ctrl BJ cells were seeded both at a density of 15.000 cells per coverslip (Sarstedt, cat #83.1840.002) and incubated overnight in a cell culture incubator (5% Oxygen). Next day, cells have been washed with PBS and fixed in 4% PFA/PBS. Cells were stored at 4° C. until IF.

5 wt % BSA (Sigma) in PBS was used as blocking buffer and antibody diluent. 1 hr blocking was followed by incubation overnight at 4° C. using a rabbit-anti-human TSPAN13 antibody (Genetex, cat #52155) as a primary antibody (dilution 1:50 in antibody diluent). Secondary antibody used was a goat-anti-rabbit-AlexaFluor488 (Thermo Fisher Scientific, cat # R37116). Incubation for 90 minutes in the dark with gentle agitation. After 3 washes with PBS and 1 wash with MilliQ water, the coverslips were mounted onto glass slides using ProLong Gold Antifade Mountant with DAPI (Thermo Fisher Scientific, cat # P36941). Images were made using a Leica DMI6000.

### FACS Analysis of TSPAN13 Expression (FIG. 18)

ctrl BJ cells and 8d IR BJ cells were fixed in 70% ethanol and stored in 4° C. until FACS analysis. All further incubations were performed at 4° C. FACS buffer used is 1% BSA in PBS. All wash steps are in 400 μl FACS buffer, 5 minutes at 800×g, 4 degrees Celsius. 1 million ctrl BJ cells and 600.000 8d IR BJ cells were used for FACS analysis. Primary antibody used is rabbit-anti-human TSPAN13 antibody (Genetex, cat #52155, dilution 1:20 in FACS buffer). Incubation for 1 hr with primary antibody. 3 washing steps were performed in between primary and secondary antibody incubations. Secondary antibody used was goat-anti-rabbit-AlexaFluor488 (Thermo Fisher Scientific, cat # R37116). Incubation for 30 minutes in the dark. Three washing steps were performed before cells were transferred through the cell strainer caps into the FACS tubes (Corning, cat #352235). Fluorescent signals were measured using a BD FACS CANTO II. FACS data analysis was performed using Kaluza software (Beckman Coulter).

### TSPAN13—Cell Membrane Expression (FIG. 19)

ctrl WI-38 cells, 7d Palbociclib (after daily treatment with 1 uM) and 7d doxorubicin treated WI-38 cells were used for analyzing TSPAN13 expression on the cell membrane via FACS. After harvesting, the cells were washed with PBS and a subsequent wash in FACS buffer (1% BSA and 0.1% NaN₃ in PBS). All wash steps were in 400 μl FACS buffer, 3 minutes at 800×g, 4° C. All further incubations were performed at 4° C. unless stated otherwise. Primary antibody used was rabbit-anti-human TSPAN13 antibody (Genetex, cat #52155, dilution 1:50 in FACS buffer): incubation for 30 minutes. Cells were washed with FACS buffer and fixed in 4% paraformaldehyde (Thermo Fisher Scientific, cat #28908) in PBS for 15 minutes at room temperature. After 3 washes with FACS buffer, cells were stored in PBS at 4° C. Secondary antibody incubation was performed using goat-anti-rabbit-AF633 (Thermo Fisher Scientific, cat # A21070) for 30 minutes. Three wash steps were performed before cells were transferred through the cell strainer caps into the FACS tubes (Corning, cat #352235). Fluorescent signals were measured using a BD FACS CANTO II. FACS data analysis was performed using Kaluza software (Beckman Coulter).

### Sorting TSPAN13 Expressing Cells (FIG. 20)

ctrl BJ cells and 9d IR BJ cells were used for sorting cells with TSPAN13 expression, representing a positive population (cell membrane staining of TSPAN13) vs. a TSPAN13 negative population. Cells were not fixed, and sorted for TSPAN13 expression on the same day as the harvest of the cells. FACS Buffer was 1% BSA in PBS and wash steps are in FACS buffer: 3 minutes at 800×g, 4° C. All incubations were performed at 4° C. Primary antibody used was rabbit-anti-human TSPAN13 antibody (Genetex, cat #52155, dilution 1:50 in FACS buffer): incubation for 1 hr. 3 Washes with FACS buffer before secondary antibody incubation using goat-anti-rabbit-AlexaFluor488 (Thermo Fisher Scientific, cat # R37116). Incubation for 30 minutes in the dark. Three washing steps were performed before cells were transferred through the cell strainer caps into the FACS tubes (Corning, cat #352235). Fluorescent signals were measured and cells were sorted into RNA lysis buffer from the ISOLATE II RNA Mini Kit (Bioline, cat #52073) using a BD FACS JAZZ. FACS data analysis was performed using Kaluza software (Beckman Coulter). RNA isolation was performed according to the manual. Reverse transcriptase PCR was performed using the High-Capacity cDNA Reverse Transcription Kit (Applied Biosystems, cat #4368813). qPCR for 2 cell cycle arrest genes (p16 and p21) and for TSPAN13 was performed using a LC480 (Roche) and SensiFAST Probe Lo-ROX Kit (Bioline, cat #84020). Tubulin was used as a reference gene. Primers: p16, F-GAGCAGCATGGAGCCTTC, R-CGTAACTATTCGGTGCGTTG, UPL probe #67; p21, F-TCACTGTCTTGTACCCTTGTGC, R-GGCGTTTGGAGTGGTAGAAA, UPL probe #32; TSPAN13, F-CCCTCAACCTGCTTTACACC, R-AATCAGCCCGAAGCCAAT, UPL probe #84; Tubulin, F-CTTCGTCTCCGCCATCAG, R-CGTGTTCCAGGCAGTAGAGC, UPL probe #40;

1. A biomarker panel comprising six or more polypeptides, or their encoding mRNA's, wherein the panel comprises at least the biomarkers TSPAN13, GDNF, C2CD5, SUSD6, BCL2L2, and PLK3, or a variant or fragment of each, as a biomarker set for cellular senescence.

2. The panel according to claim 1, wherein the set further comprises one or more biomarkers selected from the group consisting of CTLN, FAM214B, PATZ 1, PLXNA3, STAG1, TOLLIP, TRDMT1, ZBTB7A, ARID2, B4GALT7, CHMP5, CREBBP, DDA1, DYNLT3, EFNB3, ICE1, MEIS1, NOL3, PCIF1, PDLIM4, PDS5B, RAI14, RHNO1, SCOC, SLC16A3, SMO, SPIN4, TAF13, TMEM87B, UFM1, and ZNHIT1, or a variant or fragment of each.

3. The panel according to claim 1, wherein the set comprises the biomarkers TSPAN13, GDNF, C2CD5, SUSD6, BCL2L2, PLK3, and one or both of DYNLT3 and PLXNA3, or a variant or fragment of each.

4. The panel according to claim 3, wherein the set consists of the biomarkers TSPAN13, GDNF, C2CD5, SUSD6, BCL2L2, PLK3, DYNLT3 and PLXNA3, or a variant or fragment of each.

5. A method for detecting cellular senescence, wherein said method comprises the use of TSPAN13 polypeptide or its encoding mRNA, or a variant or fragment thereof, as a biomarker for cellular senescence.

6. A method of detecting a senescent cell in a test sample, the method comprising detecting the expression, in the sample, of at least the biomarker panel of claim 1, wherein an altered level of expression of at least one of said biomarkers relative to the level of expression detected in a reference sample is an indication of a senescent cell present in the sample.

7. The method according to claim 5, wherein an increased level of expression of TSPAN13, GDNF, C2CD5, PLXNA3, SUSD6, BCL2L2 and/or PLK3, or a variant or fragment thereof, relative to the level of expression detected in a reference sample is an indication of a senescent cell present in the sample.

8. The method according to claim 6, wherein the test sample is a bodily sample taken from a test subject, wherein the sample comprises blood, plasma, serum, spinal fluid, urine, sweat, saliva, tears, breast aspirate, prostate fluid, seminal fluid, vaginal fluid, stool, cervical scraping, cytes, amniotic fluid, intraocular fluid, mucous, moisture in breath, animal tissue, cell lysates, tumour tissue, hair, skin, buccal scrapings, nails, bone marrow, cartilage, prions, bone powder, ear wax, or a combination thereof.

9. (canceled)

10. A senescent cell detection kit for detecting senescent cells in a sample, the kit comprising means for detecting the presence, in a sample from a test subject, of at least the biomarker panel of claim 1.

11. The kit according to either claim 10, wherein the kit comprises at least one control or reference sample.

12. A drug conjugate for killing a senescent cell, the conjugate comprising (i) a senescent cell targeting agent configured, in use, to specifically target and bind to at least one senescent cell biomarker selected from the group consisting of TSPAN13, GDNF, FAM214B, PLXNA3, SUSD6, TOLLIP, ZBTB7A, B4GALT7, BCL2L2, CHMP5, DDA1, DYNLT3, NOL3, PDLIM4, PLK3, RAI14, SCOC, SLC16A3, TAF13, TMEM87B, UFM1 and ZNHIT1, and (ii) a cytotoxic agent, which kills the bound senescent cell.

13. The drug conjugate according to claim 12, comprising a targeting agent configured, when in use, to specifically target and bind to TSPAN13.

14. The drug conjugate according to claim 12, wherein the targeting agent is an antibody or an antigen binding fragment thereof, an aptamer, a plastic antibody or a small molecule.

15. The drug conjugate according to claim 12, wherein the cytotoxic agent is a senolytic agent, a radioisotope, a toxin or a toxic peptide.

16. The drug conjugate according to claim 15, wherein the senolytic is (a) an inhibitor of a Bcl-2 anti-apoptotic protein family member; (b) an MDM2 inhibitor; or (c) an Akt specific inhibitor.

17. (canceled)

18. A method for treating, postponing, preventing or ameliorating atherosclerosis, cardiovascular disease, cancer, arthritis, glaucoma, cataracts, osteoporosis, type 2 diabetes, hypertension, or Alzheimer's disease or other type of dementia, wherein the method comprises administering, to a subject, the drug conjugate according to claim 12.

19. A pharmaceutical composition comprising a drug conjugate according to claim 12, and a pharmaceutically acceptable vehicle.

20. A method for treating a senescence-associated disease or disorder comprising administering to a subject in need thereof a pharmaceutical composition comprising a therapeutically-effective amount of a drug conjugate according to claim 12 that selectively kills senescent cells over non-senescent cells.

21. The drug conjugate, according to claim 12, wherein the senescent cell targeting agent is configured to, in use, specifically target and bind to at least one senescent cell biomarker selected from the group consisting of TSPAN13, PLXNA3, SUSD6, GDNF, FAM214B, TOLLIP and ZBTB7A.

22. The drug conjugate, according to claim 16, wherein the inhibitor is selected from ABT-263, ABT-737, WEHI-539, and A-l 155463.
